(19) United States
(12) Patent Application Publication
(10) Pub. No.: US 2018/0289665 A1
(43) Pub. Date: Oct. 11, 2018

(54) USE OF CANNABIS TO TREAT MIGRAINE

(71) Applicant: ONE WORLD CANNABIS LTD, PETACH TIKVA (IL)

(72) Inventors: Ziv TURNER, PETACH TIKVA (IL); Alon SINAI, PETACH TIKVA (IL); Yehuda BARUCH, GEDERA (IL)

(73) Assignee: ONE WORLD CANNABIS LTD, PETACH TIKVA (IL)

(21) Appl. No.: 15/533,684

(22) PCT Filed: Dec. 7, 2015
(86) PCT No.: PCT/IL2015/051184

§ 371 Date: Jun. 7, 2017

Related U.S. Application Data
(60) Provisional application No. 62/088,599, filed on Dec. 7, 2014.

(51) Int. Cl. A61K 31/352; A61K 31/05; A61K 36/185; A61K 45/06; A61P 25/00; A61K 9/20

(52) U.S. Cl. CPC A61K 31/352; A61K 31/05; A61K 36/185; A61K 45/06; A61P 25/00; A61K 9/2095; A61K 9/2009; A61K 9/2013; A61K 9/2018; A61K 9/2059; A61K 9/2054; A61K 9/2027; A61K 9/2068

(57) ABSTRACT

The present invention discloses a composition comprising a therapeutically effective amount of Tetrahydrocannabinol (THC) or a derivative thereof, or Cannabidiol (CBD) or a derivative thereof, or a combination thereof for use in relieving migraine attack of a patient. The present invention further discloses methods for relieving migraine attack symptoms using the afore-mentioned composition.

### FIELD OF THE INVENTION

The present disclosure relates to novel compositions and methods for treatment of migraine. More particularly the current invention pertains to a composition comprising Tetrahydrocannabinol (THC), Cannabidiol (CBD) or derivatives thereof for treating acute migraine attack and methods thereof.

### BACKGROUND OF THE INVENTION

Migraine is a common, disabling headache disorder, ranked seventh highest among specific causes of disability globally (Steiner T S, Stovner L J, Birbeck G L. Migraine: the seventh disabler. Journal of Headache and Pain 2013;14:1), and with considerable social and economic impact (Hazard E, Munakata J, Bigal M E, Rupnow M F, Lipton R B. The burden of migraine in the United States: current and emerging perspectives on disease management and economic analysis. Value in Health 2009;12(1):55-64). Recent reviews found a one-year prevalence of 15% globally (Vos T, Flaxman A D, Naghavi M, Lozano R, Michaud C, Ezzati M, et al. Years lived with disability (YLDs) for 1160 sequelae of 289 diseases and injuries 1990-2010: a systematic analysis for the Global Burden of Disease Study 2010. Lancet 2012;380(9859):2163-96) and for adults in European countries (Stovner L J, Andree C. Prevalence of headache in Europe: a review for the Eurolight project. Journal of Headache and Pain 2010;11(4):289-99), 13% for all ages in the USA (Victor T W, Hu X, Campbell J C, Buse D C, Lipton R B. Migraine prevalence by age and sex in the United States: a life-span study. Cephalalgia 2010;30(9):1065-72), 21% in Russia (Ayzenberg I, Katsarava Z, Sborowski A, Chernysh M, Osipova V, Tabeeva G, et al. The prevalence of primary headache disorders in Russia: a countrywide survey. Cephalalgia 2012;32(5):373-81), and 9% for adults in China (Yu S, Liu R, Zhao G, Yang X, Qiao X, Feng J, et al. The prevalence and burden of primary headaches in China: a population-based door-to-door survey. Headache 2012;52(4):582-91). Migraine is more prevalent in women than in men (by a factor of two to three), and in the age range 30 to 50 years.

Acute treatment for each migraine episode is managed with abortive medications. Abortive medications include triptans, nonsteroidal anti-inflammatory drugs (NSAIDs), acetaminophen, ergotamine tartrate, and dihydroergotamine (Silberstein S D. Practice parameter: Evidence-based guidelines for migraine headache (an evidence-based review): Report of the Quality Standards Subcommittee of the American Academy of Neurology. Neurology. 2000;55:754-762). Nearly all (98%) migraineurs used acute treatments for attacks, with 49% using over-the-counter (OTC) medication only, 20% using prescription medication, and 29% using both. OTC medications included aspirin, other non-steroidal anti-inflammatory drugs (NSAIDs), paracetamol (acetaminophen), and paracetamol plus caffeine (Bigal M E, Serrano D, Reed M, Lipton R B. Chronic migraine in the population: burden, diagnosis, and satisfaction with treatment. Neurology 2008;71(8):559-66; Diamond S, Bigal M E, Silberstein S, Loder E, Reed M, Lipton R B. Patterns of diagnosis and acute and preventive treatment for migraine in the United States: results from the American Migraine Prevalence and Prevention study. Headache 2007;47(3):355-63; Lipton R B, Bigal M E, Diamond M, Freitag F, Reed M L. Migraine prevalence, disease burden, and the need for preventive therapy. Neurology 2007;68(5):342-9). Similar findings have been reported from other large studies in France and Germany (Lucas C, Géraud G, Valade D, Chautard M H, Lantéri-Minet M. Recognition and therapeutic management of migraine in 2004, in France: results of FRAMIG 3, a French nationwide population-based survey. Headache 2006;46(5):715-25; Radtke A, Neuhauser H. Prevalence and burden of headache and migraine in Germany. Headache 2009;49(1):79-89). Successful treatment of acute migraine attacks not only benefits patients by reducing their disability and improving health-related quality of life, but also reduces the need for healthcare resources and increases economic productivity (Jhingran P, Cady R K, Rubino J, Miller D, Grice R B, Gutterman D L. Improvements in health-related quality of life with sumatriptan treatment for migraine. Journal of Family Practice 1996;42(1):36-42; Lofland J H, Johnson N E, Batenhorst A S, Nash D B. Changes in resource use and outcomes for patients with migraine treated with sumatriptan: a managed care perspective. Archives of Internal Medicine 1999;159(8):857-63).

The acute treatment of migraine was revolutionized in the early 1990s with the introduction of sumatriptan, the first medication in the triptan class. Despite the specificity, efficacy, and safety of this class of medications, many migraineurs do not respond optimally or at all to triptans, or have contraindications to their use. Of those who do respond, a significant number are unable to tolerate the adverse effects or do not experience complete and sustained pain relief (Ferrari M D, Roon K I, Lipton R B, Goadsby P J. Oral triptans (serotonin 5-HT (1B/1D) agonists) in acute migraine treatment: a meta-analysis of 53 trials. Lancet. 2001;358(9294):16). Therefore despite recent advances, adequate headache pain, disability, and associated symptom relief continues to elude millions of migraine and cluster headache sufferers.

The study of cannabinoids for pain only started in the early 1970s. Patient self-report surveys on marijuana have been positive for a variety of symptoms including headache (Schnelle M, Grotenhermen F, Reif M, Gorter R W. Ergebnisse einer standardisierten Umfrage zur medizinischen Verwendung von Cannabisprodukten im deutschen Sprachraum, [Results of a standardized survey on the medical use of cannabis products in the German-speaking area] [Research in Complementary Medicine]. Forsch Komplementarmed. 1999;6(Suppl. 3):28-36). El-Mallakh described 3 long-term daily marijuana users who developed headache after cessation of marijuana, matching clinical experience of headache being a symptom of withdrawal (El-Mallakh R S. Marijuana and migraine. Headache. 1987;27:442-443). Surveys on 2480 patients of the Oakland Cannabis Buyer's Club by Mikuriya indicate 5% used cannabis for relief of migraine (Gieringer D. Medical use of cannabis: Experience in California. In: Grotenhermen F, Russo E B, eds. Cannabis and Cannabinoids: Pharmacology, Toxicology and Therapeutic Potential. Binghamton, N.Y.: The Hawthorn Press, Inc; 2002:143-152).

In view of the above, there is still a long felt and unmet need for a naturally originated composition with minimal adverse effects that is specifically useful for treatment of migraine.

### SUMMARY OF THE INVENTION

It is thus one object of the present invention to disclose a composition comprising a therapeutically effective amount of Tetrahydrocannabinol (THC) or a derivative thereof, or Cannabidiol (CBD) or a derivative thereof, or a combination thereof for use in relieving migraine attack of a patient.

It is a further object of the present invention to disclose the composition as defined above, wherein said CBD and said THC are in a predefined ratio conferring relief of migraine attack symptoms.

It is a further object of the present invention to disclose the composition as defined in any of the above, wherein said CBD and said THC are in a predefined ratio conferring an additive effect with respect to relieving of migraine attack symptoms relative to the effect conferred by said CBD and said THC administered separately in a similar concentration.

It is a further object of the present invention to disclose the composition as defined in any of the above, wherein said CBD and said THC are in a predefined ratio conferring a synergistic effect with respect to relieving of migraine attack symptoms relative to the effect conferred by said CBD and said THC administered separately in a similar concentration.

It is a further object of the present invention to disclose the composition as defined in any of the above, wherein said predefined ratio of said CBD and said THC is selected from the group consisting of about 1:1, 1:5, 5:1, 1:4 and 4:1, respectively.

It is a further object of the present invention to disclose the composition as defined in any of the above, wherein said symptoms are selected from the group consisting of nausea, vomiting, sensitivity to light, sensitivity to sound, sensitivity to smell and any combination thereof.

It is a further object of the present invention to disclose the composition as defined in any of the above, wherein said THC or a derivative thereof is selected from the group consisting of THC, THCV, THCA, THCVA and any combination thereof.

It is a further object of the present invention to disclose the composition as defined in any of the above, wherein said THC or a derivative thereof is selected from the group consisting of natural THC or a derivative thereof produced in the body of humans and animals, THC or a derivative thereof extracted from plants, synthetic THC or a derivative thereof, and any combination thereof.

It is a further object of the present invention to disclose the composition as defined in any of the above, wherein said THC or a derivative thereof is extracted from cannabis plant; said cannabis plant is selected from a group consisting of: Cannabis sativa, Cannabis indica, Cannabis ruderalis, and any combination thereof.

It is a further object of the present invention to disclose the composition as defined in any of the above, wherein said composition provides a synergistic effect with respect to relieving acute migraine attack as compared to the effect provided by THC or a derivative thereof or by CBD or a derivative thereof administered separately in a similar concentration.

It is a further object of the present invention to disclose the composition as defined in any of the above, wherein said Cannabidiol (CBD) or a derivative thereof is selected from the group consisting of CBD, CBDV, CBDA and any combination thereof.

It is a further object of the present invention to disclose the composition as defined in any of the above, wherein said CBD or a derivative thereof is selected from the group consisting of natural CBD or a derivative thereof produced in the body of humans and animals, CBD or a derivative thereof extracted from plants, synthetic CBD or a derivative thereof, and any combination thereof.

It is a further object of the present invention to disclose the composition as defined in any of the above, wherein said CBD or a derivative thereof is extracted from cannabis plant; said cannabis plant is selected from a group consisting of: Cannabis sativa, Cannabis indica, Cannabis ruderalis, and any combination thereof.

It is a further object of the present invention to disclose the composition as defined in any of the above, wherein the concentration of said THC or a derivative thereof in said composition is in the range of about 0.2% to about 10%.

It is a further object of the present invention to disclose the composition as defined in any of the above, wherein said composition is configured for administration in a dosage unit of between about 0.5 mg to about 50 mg of said THC or a derivative thereof.

It is a further object of the present invention to disclose the composition as defined in any of the above, wherein said composition comprises between about 1 wt. % to about 10 wt. % of said THC or a derivative thereof.

It is a further object of the present invention to disclose the composition as defined in any of the above, wherein the concentration of said CBD or a derivative thereof in said composition is in the range of about 1% to about 50%.

It is a further object of the present invention to disclose the composition as defined in any of the above, wherein said composition is configured for administration in a dosage unit of between about 1 mg to about 300 mg of said CBD or a derivative thereof.

It is a further object of the present invention to disclose the composition as defined in any of the above, wherein said composition comprises between about 1 wt. % to about 30 wt. % of said CBD or a derivative thereof.

It is a further object of the present invention to disclose the composition as defined in any of the above, wherein the concentration of said CBD or said derivative thereof is in the range of about 2% (wt.) to about 20%. (wt.).

It is a further object of the present invention to disclose the composition as defined in any of the above, wherein the concentration of said THC or said derivative thereof is in the range of about 2% (wt.) to about 20% (wt.).

It is a further object of the present invention to disclose the composition as defined in any of the above, wherein said composition comprises cannabis oil.

It is a further object of the present invention to disclose the composition as defined in any of the above, wherein said cannabis oil is in a concentration of about 2% (wt.) to about 25% (wt.).

It is a further object of the present invention to disclose the composition as defined in any of the above, wherein said migraine attack is selected from the group consisting of acute migraine attack and chronic migraine attack.

It is a further object of the present invention to disclose the composition as defined in any of the above, wherein said composition provides an improvement in migraine attack symptoms of said patient measured by at least one pain severity scale, compared to an established baseline or placebo.

It is a further object of the present invention to disclose the composition as defined in any of the above, wherein said improvement in migraine attack symptoms of said patient is measured by a decrease in the patient's score of at least one point or level on said at least one pain severity scale, as compared to an established baseline or to a placebo.

It is a further object of the present invention to disclose the composition as defined in any of the above, wherein said improvement in migraine attack symptoms of said patient is measured by a decrease in the patient's score of at least two points at the first two hours from the administration of said composition to said patient, on said at least one pain severity scale, as compared to an established baseline or to a placebo.

It is a further object of the present invention to disclose the composition as defined in any of the above, wherein said pain severity scale is selected from a group consisting of four point pain severity scale, Chronic Migraine Severity Scale, Comparative Pain Scale, Wong baker scale, The Chronic Pain Index, the Migraine Disability Assessment (MIDAS), the Headache Impact Test (HIT), the Visual Analogue Scale (VAS), the 4-point verbal rating scale (VRS), the 6-point VRS, the Numerical rating scales (NRS), the Faces Pain Rating Scale and any combination thereof.

It is a further object of the present invention to disclose the composition as defined in any of the above, wherein said symptoms are selected from a group consisting of nausea, vomiting, sensitivity to light, sensitivity to sound, sensitivity to smell and any combination thereof.

It is a further object of the present invention to disclose the composition as defined in any of the above, wherein said composition is formulated for an administration route selected from the group consisting of: intranasal, transdermal, intravenous, vaginal, sublingual, buccal, oral, and any combination thereof.

It is a further object of the present invention to disclose the composition as defined in any of the above, wherein said composition is formulated in a dosage form selected from the group consisting of liquid, solid, gas, oral, sublingual, pill, tablet, capsule, buccal, tincture, strip, film, spray, lozenge, effervescent form, sub-lingual, granules, orally-disintegrating, thin film, liquid, solution, suspension, emulsion, powder or liquid or solid crystals, powder, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, syrup, skin patch, vaginal, suppository, pessary, rectal and any combination thereof.

It is a further object of the present invention to disclose the composition as defined in any of the above, wherein said composition is administered in combination with at least one migraine therapeutic agent.

It is a further object of the present invention to disclose the composition as defined in any of the above, wherein said at least one migraine therapeutic agent is selected from a group consisting of pain-relieving medications, aspirin, acetaminophen, indomethacin, Ergots, anti-nausea medications, NSAID, triptan, Opioid medications, Glucocorticoids such as prednisone and dexamethasone, Cardiovascular drugs, Antidepressants, Anti-seizure drugs, OnabotulinumtoxinA (Botox), Pain relievers and any combination thereof.

It is a further object of the present invention to disclose the composition as defined in any of the above, wherein said composition combined with at least one migraine therapeutic agent provides a synergistic effect with respect to relieving migraine attack symptoms relative to the effect provided by said migraine therapeutic agent administered separately.

It is a further object of the present invention to disclose the composition as defined in any of the above, wherein said composition additionally comprises at least one carrier or excipient selected from the group consisting of diluent, solvent, absorbent, anti-adherent, binder, coatings, disintegrant, surfactant, dissolving agent, solubilizing agent, bioadhesive agent, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents, glidant, flavours, colours, sweetener, thickener, lubricant, sorbents, preservatives, and any combination thereof.

It is a further object of the present invention to disclose the composition as defined in any of the above, wherein said solvent is ethanol.

It is a further object of the present invention to disclose the composition as defined in any of the above, wherein said composition is substantially free of a pharmaceutically acceptable emulsifying agent or surfactant.

It is a further object of the present invention to disclose the composition as defined in any of the above, wherein said composition is formulated in a sublingual dosage form.

It is a further object of the present invention to disclose the composition as defined in any of the above, wherein said composition is formulated in a solid dosage form.

It is a further object of the present invention to disclose the composition as defined in any of the above, wherein said THC and said CBD are formulated for penetrating the mucosal barrier.

It is a further object of the present invention to disclose the composition as defined in any of the above, wherein said composition is formulated for rapid disintegration upon administration.

It is a further object of the present invention to disclose the composition as defined in any of the above, wherein said composition is not significantly psychoactive.

It is a further object of the present invention to disclose the composition as defined in any of the above, wherein said composition is in a sustained release dosage form or in an immediate release dosage form.

It is a further object of the present invention to disclose the composition as defined in any of the above, wherein said sustained release dosage form is selected from the group consisting of liposomes, drug polymer conjugates, microencapsulation, controlled-release tablet coating, and any combination thereof.

It is a further object of the present invention to disclose the composition as defined in any of the above, wherein said composition is in an effervescent form.

It is a further object of the present invention to disclose the composition as defined in any of the above, wherein said composition is administered once, twice, three or four times through the day.

It is a further object of the present invention to disclose the composition as defined in any of the above, wherein administration of said composition to a patient suffering from migraine attack increases cerebral blood flow (CBF) as measure by functional magnetic resonance imaging (fMRI), in comparison with the cerebral blood flow (CBF) of said patient prior to the administration of said composition to said patient or as compared to a healthy control.

It is a further object of the present invention to disclose the composition as defined in any of the above, wherein said fMRI is selected from the group consisting of: diffusion-weighted imaging (DWI), perfusion-weighted imaging (PWI), blood oxygenation level-dependent (BOLD) imaging, and any combination thereof.

It is a further object of the present invention to disclose the composition as defined in any of the above, wherein administration of said composition to a patient suffering from migraine attack, reduces paroxysmal cortical neuron depolarization as measure by electroencephalogram (EEG) or functional magnetic resonance imaging (fMRI), in comparison with the paroxysmal cortical neuron depolarization of said patient prior to the administration of said composition to said patient or as compared to a healthy control.

It is a further object of the present invention to disclose the composition as defined in any of the above, wherein said composition is dissolved in a lipophilic solvent or suspension carrier.

It is a further object of the present invention to disclose the composition as defined in any of the above, wherein said lipophilic solvent or suspension carrier are selected from a group consisting of ethanol, medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

It is a further object of the present invention to disclose a synergistically effective composition, wherein said composition comprising a therapeutically effective amount of Cannabidiol (CBD) or a derivative thereof and Tetrahydrocannabinol (THC) or a derivative thereof in a predefined ratio conferring a synergistic effect with respect to relieving migraine attack of a patient, relative to the effect of said CBD and said THC administered separately in a similar concentration.

It is a further object of the present invention to disclose the synergistically effective composition as defined above, wherein said predefined ratio of said CBD and said THC is selected from the group consisting of: about 1:1, 5:1, 1:5, 1:4 and 4:1 respectively.

It is a further object of the present invention to disclose a method for relieving migraine attack of a patient comprising steps of (a) providing a composition comprising Tetrahydrocannabinol (THC) or a derivative thereof, or Cannabidiol (CBD) or a derivative thereof, or a combination thereof; and (b) administering said composition to said patient at a therapeutically effective dosage for relieving migraine attack of said patient.

It is a further object of the present invention to disclose the method as defined above, additionally comprising steps of providing said composition comprising said CBD and said THC in a predefined ratio conferring relief of migraine attack symptoms.

It is a further object of the present invention to disclose the method as defined in any of the above, wherein said CBD and said THC are in a predefined ratio conferring an additive effect with respect to relieving of migraine attack symptoms relative to the effect conferred by said CBD and said THC administered separately in a similar concentration.

It is a further object of the present invention to disclose the method as defined in any of the above, wherein said CBD and said THC are in a predefined ratio conferring a synergistic effect with respect to relieving of migraine attack symptoms relative to the effect conferred by said CBD and said THC administered separately in a similar concentration.

It is a further object of the present invention to disclose the method as defined in any of the above, additionally comprising steps of selecting said predefined ratio of said CBD and said THC from the group consisting of about 1:1, 1:5, 5:1, 1:4 and 4:1, respectively.

It is a further object of the present invention to disclose the method as defined in any of the above, additionally comprising steps of selecting said symptoms from the group consisting of nausea, vomiting, sensitivity to light, sensitivity to sound, sensitivity to smell and any combination thereof.

It is a further object of the present invention to disclose the method as defined in any of the above, additionally comprising steps of selecting said THC or a derivative thereof from the group consisting of THC, THCV, THCA, THCVA and any combination thereof.

It is a further object of the present invention to disclose the method as defined in any of the above, additionally comprising steps of selecting said THC or a derivative thereof from the group consisting of natural THC or a derivative thereof produced in the body of humans and animals, THC or a derivative thereof extracted from plants, synthetic THC or a derivative thereof, and any combination thereof.

It is a further object of the present invention to disclose the method as defined in any of the above, additionally comprising steps of extracting said THC or a derivative thereof from cannabis; said cannabis is selected from a group consisting of: Cannabis sativa, Cannabis indica, Cannabis ruderalis, and any combination thereof.

It is a further object of the present invention to disclose the method as defined in any of the above, additionally comprising steps of providing a synergistic effect with respect to relieving acute migraine attack as compared to the effect provided by said THC or a derivative thereof or by said CBD or a derivative thereof administered separately in a similar concentration.

It is a further object of the present invention to disclose the method as defined in any of the above, additionally comprising steps of selecting said Cannabidiol (CBD) or a derivative thereof from the group consisting of CBD, CBDV, CBDA and any combination thereof.

It is a further object of the present invention to disclose the method as defined in any of the above, additionally comprising steps of selecting said CBD or a derivative thereof from the group consisting of natural CBD or a derivative thereof produced in the body of humans and animals, CBD or a derivative thereof extracted from plants, synthetic CBD or a derivative thereof, and any combination thereof.

It is a further object of the present invention to disclose the method as defined in any of the above, additionally comprising steps of extracting said CBD or a derivative thereof from cannabis; said cannabis is selected from a group consisting of: Cannabis sativa, Cannabis indica, Cannabis ruderalis, and any combination thereof.

It is a further object of the present invention to disclose the method as defined in any of the above, additionally comprising steps of providing said THC or a derivative thereof in a concentration in the range of about 0.2% to about 10%.

It is a further object of the present invention to disclose the method as defined in any of the above, additionally comprising steps of providing said composition configured for administration in a dosage unit of between about 0.5 mg to about 50 mg of said THC or a derivative thereof.

It is a further object of the present invention to disclose the method as defined in any of the above, additionally comprising steps of formulating said composition comprising between about 1 wt. % to about 10 wt. % of said THC or a derivative thereof.

It is a further object of the present invention to disclose the method as defined in any of the above, additionally comprising steps of formulating said composition comprising CBD or a derivative thereof concentration in the range of about 1% to about 50%.

It is a further object of the present invention to disclose the method as defined in any of the above, additionally comprising steps of formulating said composition configured for administration in a dosage unit of between about 1 mg to about 300 mg of said CBD or a derivative thereof.

It is a further object of the present invention to disclose the method as defined in any of the above, additionally comprising steps of formulating said composition comprising between about 1 wt. % to about 30 wt. % of said CBD or a derivative thereof.

It is a further object of the present invention to disclose the method as defined in any of the above, wherein the concentration of said CBD or said derivative thereof is in the range of about 2% (wt.) to about 20%. (wt.).

It is a further object of the present invention to disclose the method as defined in any of the above, wherein the concentration of said THC or said derivative thereof is in the range of about 2% (wt.) to about 20% (wt.).

It is a further object of the present invention to disclose the method as defined in any of the above, wherein said composition comprises cannabis oil.

It is a further object of the present invention to disclose the method as defined in any of the above, wherein said cannabis oil is in a concentration of about 2% (wt.) to about 25% (wt.).

It is a further object of the present invention to disclose the method as defined in any of the above, additionally comprising steps of selecting said migraine attack from the group consisting of acute migraine attack and chronic migraine attack.

It is a further object of the present invention to disclose the method as defined in any of the above, additionally comprising steps of administering said composition to said patient at a therapeutically effective dosage so as to provide an improvement in migraine attack symptoms of said patient as measured by at least one pain severity scale, compared to an established baseline or placebo.

It is a further object of the present invention to disclose the method as defined in any of the above, additionally comprising steps of administering said composition to said patient at a therapeutically effective dosage so as to provide an improvement in migraine attack symptoms of said patient as measured by a decrease in the patient's score of at least one point or level on said at least one pain severity scale, as compared to an established baseline or to a placebo.

It is a further object of the present invention to disclose the method as defined in any of the above, additionally comprising steps of administering said composition to said patient at a therapeutically effective dosage so as to provide an improvement in migraine attack symptoms of said patient as measured by a decrease in the patient's score of at least two points or levels at the first two hours from administration of said composition to said patient, on said at least one pain severity scale, as compared to an established baseline or to a placebo.

It is a further object of the present invention to disclose the method as defined in any of the above, additionally comprising steps of selecting said pain severity scale from a group consisting of four point pain severity scale, Chronic Migraine Severity Scale, Comparative Pain Scale, Wong baker scale, The Chronic Pain Index, the Migraine Disability Assessment (MIDAS), the Headache Impact Test (HIT), the Visual Analogue Scale (VAS), the 4-point verbal rating scale (VRS), the 6-point VRS, the Numerical rating scales (NRS) and the Faces Pain Rating Scale and any combination thereof.

It is a further object of the present invention to disclose the method as defined in any of the above, additionally comprising steps of selecting said symptoms from the group consisting of nausea, vomiting, sensitivity to light, sensitivity to sound, sensitivity to smell and any combination thereof.

It is a further object of the present invention to disclose the method as defined in any of the above, additionally comprising steps of formulating said composition for an administration route selected from the group consisting of: intranasal, transdermal, intravenous, vaginal, sublingual, buccal, oral, and any combination thereof.

It is a further object of the present invention to disclose the method as defined in any of the above, additionally comprising steps of formulating said composition in a dosage form selected from the group consisting of liquid, solid, gas, oral, sublingual, pill, tablet, capsule, buccal, tincture, strip, film, spray, lozenge, effervescent form, sub-lingual, granules, orally-disintegrating, thin film, liquid, solution, suspension, emulsion, powder or liquid or solid crystals, powder, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, syrup, skin patch, vaginal, suppository, pessary, rectal and any combination thereof.

It is a further object of the present invention to disclose the method as defined in any of the above, additionally comprising steps of administering said composition in combination with at least one migraine therapeutic agent.

It is a further object of the present invention to disclose the method as defined in any of the above, additionally comprising steps of selecting said at least one migraine therapeutic agent from a group consisting of pain-relieving medications, aspirin, acetaminophen, indomethacin, Ergots, anti-nausea medications, NSAID, triptan, Opioid medications, Glucocorticoids such as prednisone and dexamethasone, Cardiovascular drugs, Antidepressants, Anti-seizure drugs, OnabotulinumtoxinA (Botox), Pain relievers and any combination thereof.

It is a further object of the present invention to disclose the method as defined in any of the above, additionally comprising steps of providing a synergistic effect with respect to relieving migraine attack symptoms relative to the effect provided by said migraine therapeutic agent administered separately in a similar concentration.

It is a further object of the present invention to disclose the method as defined in any of the above, additionally comprising steps of formulating said composition with at least one carrier or excipient selected from the group consisting of diluent, solvent, absorbent, anti-adherent, binder, coatings, disintegrant, surfactant, dissolving agent, solubilizing agent, bioadhesive agent, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents, glidant, flavours, colours, sweetener, thickener, lubricant, sorbents, preservatives, and any combination thereof.

It is a further object of the present invention to disclose the method as defined in any of the above, wherein said solvent is ethanol.

It is a further object of the present invention to disclose the method as defined in any of the above, additionally comprising steps of formulating said composition substantially free of a pharmaceutically acceptable emulsifying agent or surfactant.

It is a further object of the present invention to disclose the method as defined in any of the above, additionally comprising steps of formulating said composition in a sublingual dosage form.

It is a further object of the present invention to disclose the method as defined in any of the above, additionally comprising steps of formulating said composition in a solid dosage form.

It is a further object of the present invention to disclose the method as defined in any of the above, additionally comprising steps of formulating said composition for penetrating the mucosal barrier.

It is a further object of the present invention to disclose the method as defined in any of the above, additionally comprising steps of formulating said composition for rapid disintegration upon administration.

It is a further object of the present invention to disclose the method as defined in any of the above, wherein said composition is not significantly psychoactive.

It is a further object of the present invention to disclose the method as defined in any of the above, additionally comprising steps of formulating said composition in a sustained release dosage form or in an immediate release dosage form.

It is a further object of the present invention to disclose the method as defined in any of the above, additionally comprising steps of selecting said sustained release dosage form from a group consisting of liposomes, drug polymer conjugates, microencapsulation, controlled-release tablet coating, and any combination thereof.

It is a further object of the present invention to disclose the method as defined in any of the above, additionally comprising steps of administering said composition once, twice, three or four times through the day.

It is a further object of the present invention to disclose a method for increasing cerebral blood flow (CBF) in a patient suffering from migraine attack, said method comprises steps of: (a) administering a composition comprising Tetrahydrocannabinol (THC) or a derivative thereof, or Cannabidiol (CBD) or a derivative thereof, or a combination thereof to said patient at a therapeutically effective dosage; and (b) detecting increase in cerebral blood flow (CBF) of said patient by functional magnetic resonance imaging (fMRI) analysis.

It is a further object of the present invention to disclose the method or increasing cerebral blood flow (CBF) in a patient suffering from migraine attack as defined above, additionally comprises steps of selecting said fMRI from the group consisting of: diffusion-weighted imaging (DWI), perfusion-weighted imaging (PWI), blood oxygenation level-dependent (BOLD) imaging, and any combination thereof.

It is a further object of the present invention to disclose a method for reducing paroxysmal cortical neuron depolarization in a patient suffering from migraine attack comprising steps of (a) administering to said patient a composition comprising Tetrahydrocannabinol (THC) or a derivative thereof, or Cannabidiol (CBD) or a derivative thereof, or a combination thereof at a therapeutically effective dosage; and (b) detecting decrease in paroxysmal cortical neuron depolarization of said patient by electroencephalogram (EEG) or functional magnetic resonance imaging (fMRI) analysis.

It is a further object of the present invention to disclose a use of a composition comprising a therapeutically effective amount of Tetrahydrocannabinol (THC) or a derivative thereof, or Cannabidiol (CBD) or a derivative thereof, or a combination thereof in the manufacture of a medicament for relieving migraine attack of a patient.

It is a further object of the present invention to disclose the use as defined above, wherein said Tetrahydrocannabinol (THC) or a derivative thereof, or Cannabidiol (CBD) or a derivative thereof, or the combination thereof, is extracted from cannabis plant.

It is a further object of the present invention to disclose a composition comprising a therapeutically effective amount of Cannabidiol (CBD) or a derivative thereof or a Tetrahydrocannabinol (THC) or a derivative thereof for use in relieving migraine attack of a patient, wherein said composition is prepared by steps of: (a) preparing a mixture comprising an effective amount of cannabis oil, by a wet granulation process; and, (b) formulating said mixture in a solid dosage form by direct compression.

It is a further object of the present invention to disclose a composition prepared by steps as defined above, wherein said mixture is further prepared by steps of: (a) preparing a first mixture comprising said cannabis oil and a solvent; (b) preparing a second mixture comprising at least one pharmaceutically acceptable carrier or excipient selected from the group consisting of a sweetener, a disintegrant, a thickener and any combination thereof; and (c) adding said second mixture to said first mixture by mixing using a high shear granulator.

It is a further object of the present invention to disclose a composition prepared by steps as defined in any of the above, wherein said composition is further prepared by steps of: preparing said first mixture comprising cannabis oil, absorbent, lubricant and binder.

It is a further object of the present invention to disclose the composition prepared by steps as defined in any of the above, wherein said composition is further prepared by steps of: (a) drying said mixture of step c to LOD equal or less than 1%; and (b) mixing said dried mixture with at least one pharmaceutically acceptable carrier or excipient selected from the group consisting of: glidant, binder, sweetener, lubricant, disintegrant and any combination thereof.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured. The present invention provides a composition comprising THC, Cannabidiol (CBD) or derivatives thereof for treating migraine and related disorders.

The essence of the present invention is to provide a composition for treating migraine comprising at least one of Tetrahydrocannabinol (THC) or a derivative thereof, Cannabidiol (CBD) or a derivative thereof, or a combination thereof.

In 1941, Cannabis preparations were dropped from the United States Pharmacopeia (U.S.P.). However, in the following year, the editor of the Journal of the American Medical Association reported oral preparations of Cannabis in treatment of menstrual (catamenial) migraine (M Fishbein. Migraine associated with menstruation. J. Am. Med. Assoc., 237 (1942), p. 326).

It is herein acknowledged that reduced levels of anandamide (AEA) have been found in the cerebrospinal fluid of patients with chronic migraine (Sarchielli P, Pini LA, Coppola F, et al. Endocannabinoids in chronic migraine: CSF findings suggest a system failure. Neuropsychopharmacology. 2007; 32:1384-1390). Without wishing to be bound by theory, it is hypothesized that reduced inhibition from AEA leads to greater activation of the trigeminovascular system and greater propensity to migraine and longer term sensitization. The deficiency of endocannabinoids has been surprisingly shown by the present invention, to be critical in the pathophysiology of migraine and also other pain disorders (Russo E B. Clinical endocannabinoid deficiency (CECD): Can this concept explain therapeutic benefits of cannabis in migraine, fibromyalgia, irritable bowel syndrome and other treatment-resistant conditions? Neuro Endocrinol Lett. 2004; 25:31-39).

Also, platelets of female migraineurs, but not male, were recently reported to exhibit an increased activity of AEA hydrolase, suggesting accelerated endocannabinoid degradation (Cupini L M, Bari M, Battista N, et al. Biochemical changes in endocannabinoid system are expressed in platelets of female but not male migraineurs. Cephalalgia. 2006;26:277-281). Akerman and colleagues demonstrated that AEA was able to inhibit dural blood vessel dilation from electrical stimulation, administration of calcitonin gene-related peptide, nitrous oxide, and capsaicin. This effect was found to be reversed by a cannabinoid antagonist (Akerman S, Kaube H, Goadsby P J. Anandamide is able to inhibit trigeminal neurons using an in vivo model of trigeminovascular-mediated nociception. J Pharmacol Exp Ther. 2004;309:56-63).

It is therefore, within the scope of the present invention to demonstrate and provide experimental evidence such as clinical trails for the use of cannabis as an acute migraine treatment. According to some aspects, the present invention shows that the effect of cannabinoids on analgesic activity on the trigeminal nucleus caudalis, presynaptic inhibition of glutamate release, an anti-inflammatory effect, anti-emetic effect, and vasoconstrictive effect make cannabinoids a good migraine treatment, albeit with health concerns in connection with smoking and overuse.

The composition may also include cannabinoid (CBD) that is known to potentiate the activity of THC by increasing CB1 receptor density or through another CB1-related mechanism (Hayakawa et al.). CBD may increase the efficiency of the composition or provide a synergistic effect with respect to relieving migraine pain and effects as well as making it possible to use lower concentrations of THC.

As used herein the term “about” denotes ±25% of the defined amount or measure or value.

The term “Migraine” as used hereinafter generally refers to a common disease which expresses itself by paroxysmal headache, commonly accompanied by transient neurological symptoms. In further aspects, migraine is a chronic neurological disorder characterized by recurrent moderate to severe headaches often in association with a number of autonomic nervous system symptoms. Typically the headache affects one half of the head, is pulsating in nature, and lasts from 2 to 72 hours. Associated symptoms may include nausea, vomiting, and sensitivity to light, sound, or smell. Up to one-third of people with migraine headaches perceive an aura: a transient visual, sensory, language, or motor disturbance which signals that the headache will soon occur. Occasionally an aura can occur with little or no headache following it.

The term migraine as used herein also refers to acute migraine attack. The exact mechanisms of migraine are not known. It is, however, believed to be a neurovascular disorder. The main theory is related to increased excitability of the cerebral cortex and abnormal control of pain neurons in the trigeminal nucleus of the brainstem. It is within the scope of the present invention that migraines include other types of headaches, such as tension headache and sinus headache.

Without wishing to be bound by theory, currently there are two main theories concerning the cerebral mechanisms of migraine and attempted to explain migraine pathogenesis:
The vascular theory which attributed migraine primarily to cerebral artery diameter change, with constriction resulting in local hypoxia and transient focal symptoms, followed by neurogenic extracranial or intracranial vasodilation causing pain. This theory attributes migraine to spasm of a cerebral artery causing local hypoxia and transient focal symptoms followed by neurogenically mediated extra- and/or intracranial vasodilation causing headache, i.e. migraine is understood in terms of a primary perturbation of blood vessel function.
Another viewpoint regarded migraine mainly as a paroxysmal, transient depolarization of cortical neurons, resulting in focal symptoms and headache. In this theory, migraine is understood in terms of a primary perturbance of neuronal function.

It is herein acknowledged that a common migraine attack involves paroxysmal headache accompanied by focal symptoms of short duration. At the first stage of such a migraine attack, regional cerebral blood flow (rCBF) declines in the posterior part of the brain. Subsequently the hypoperfused region expands anteriorly, independent of the territories of supply of the large cerebral arteries.

The term “cannabis” refers hereinafter to a genus of flowering plants that includes three different species, Cannabis sativa, Cannabis indica and Cannabis ruderalis.

The term “Cannabinoids” refer hereinafter to a class of diverse chemical compounds that act on cannabinoid receptors on cells that repress neurotransmitter release in the brain. These receptor proteins include the endocannabinoids (endogenous ligands of the cannabinoid receptors, produced naturally in the body by humans and animals), the phytocannabinoids (found in cannabis and some other plants), and synthetic cannabinoids (manufactured chemically). The most notable cannabinoid is the phytocannabinoid Δ9-Tetrahydrocannabinol (THC), the primary psychoactive compound of cannabis. Cannabidiol (CBD) is another major constituent of the plant, representing up to 40% in extracts of the plant resin. There are at least 85 different cannabinoids isolated from cannabis, exhibiting varied effects.

The term “Cannabidiol” or “CBD” refers hereinafter to one of at least 85 active cannabinoids identified in cannabis. Cannabidiol is a major phytocannabinoid, accounting for up to 40% of the plant's extract. CBD is considered to have a wider scope of medical applications than Tetrahydrocannabinol (THC). Cannabidiol has a very low affinity for CB1 and CB2 receptors but acts as an indirect antagonist of their agonists. CBD may potentiate THC's effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion. The term CBD also refers to Cannabidivarin (CBDV) a homolog of Cannabidiol (CBD) and to Cannabidiolic acid (CBDA).

The term “Tetrahydrocannabinol” or “THC” refers hereinafter to the principal psychoactive constituent (or cannabinoid) of the cannabis plant. THC has a partial agonist activity at the cannabinoid receptor CB1, and the CB2 receptor and is known to increase cortisol levels. It is further included within the scope that the term THC further refers to Tetrahydrocannabivarin (THCV or THV) a homologue of Tetrahydrocannabinol (THC) and Tetrahydrocannabinolic acid (THCA, 2-COOH-THC), a biosynthetic precursor of Tetrahydrocannabinol (THC).

It is noted that cannabinol (CBN), cannabichromene (CBC), the acids (CBDA, CBGA, THCA) and propyl homologues (CBDV, CBGV, THCV) of CBD, cannabigerol (CBG) and THC, and Tetrahydrocannabivarin acid (THC-V and THC-VA) are also included as optional active ingredient(s) of the composition or formulation of the present invention.

The present invention further encompasses derivatives of cannabinoids (i.e. THC and/ or CBD derivatives) which include isomers of cannabinoids, synthetic derivatives of cannabinoids; in general, it includes all chemicals that bind to the cannabinoid receptors and related compounds.

The term “cannabinoid receptor” refers hereinafter to a class of cell membrane receptors under the G protein-coupled receptor superfamily. There are currently two known subtypes of cannabinoid receptors, termed CB1 and CB2. The CB1 receptor is expressed mainly in the brain, but also in the lungs, liver and kidneys. The CB2 receptor is expressed mainly in the immune system, the digestive system and in hematopoietic cells.

As used herein the term “Non Steroidal Anti-Inflammatory Drug” or “NSAID” usually abbreviated to NSAIDs or NAIDs, but also referred to as nonsteroidal anti-inflammatory agents/analgesics (NSAIAs) or nonsteroidal Anti-inflammatory medicines (NSAIMs), refers to drugs with analgesic and antipyretic effects and in higher doses have anti-inflammatory effects.

The term “nonsteroidal” is used to distinguish NSAIDs from steroids, which, among a broad range of other effects, have a similar eicosanoid-depressing, anti-inflammatory action. As analgesics, NSAIDs are unusual in that they are non-narcotic.

NSAIDs are usually indicated for the treatment of acute or chronic conditions where pain and inflammation are present. NSAIDs inhibit the activity of both cyclooxygenase-1 (COX-1) and cyclooxygenase-2 (COX-2), and thereby, the synthesis of prostaglandins and thromboxanes. Examples of NSAIDs may include acetyl salicylic acid, indometacin, sulindac, phenylbutazone, diclofenac, fentiazac, ketorolac, piroxicam, tenoxicam, mecoxicam, meloxicam, cinnoxicam, ibufenac, ibuprofen, naproxen, ketoprofen, nabumetone, niflumic acid and nimesulide, Cox-2 inhibitors or a pharmaceutically acceptable salt thereof or mixtures thereof. Other preferred NSAIDs include piroxicam, tenoxicam, mecoxicam, meloxicam, ibufenac, ibuprofen, naproxen and ketoprofen, or a pharmaceutically acceptable salt thereof, or mixtures thereof.

Useful NSAIDs can be selected from suitable acidic and nonacidic compounds. Suitable acidic compounds include carboxylic acids and enolic acids. Suitable nonacidic compounds include, for example, nabumetone, tiaramide, proquazone, bufexamac, flumizole, epirazole, tinoridine, timegadine and dapsone.

According to certain aspects of the invention, suitable carboxylic acid NSAIDs may include, salicylic acids and esters thereof, such as aspirin, diflunisal, benorylate and fosfosal; acetic acids, including phenylacetic acids such as diclofenac, alclofenac and fenclofenac, and carbo- and heterocyclic acetic acids such as etodolac, indomethacin, sulindac, rolmerin, fentiazac and tilomisole; propionic acids, such as carprofen, fenbufen, flurbiprofen, ketoprofen, oxaprozin, suprofen, tiaprofenic acid, ibuprofen, naproxen, fenoprofen, indoprofen, pirprofen; and fenamic acids, such as flufenamic, mefenamic, meclofenamic and niflumic.

According to other embodiments of the invention, suitable enolic acid NSAIDs include, for example, pyrazolones such as oxyphenbutazone, phenylbutazone, apazone and feprazone, and oxicams such as piroxicam, sudoxicam, isoxicam and tenoxicam.

The term “triptan” used herein after refers to Triptans are a family of tryptamine-based drugs used as abortive medication in the treatment of migraines and cluster headaches. None limiting examples of triptans include sumatriptan (Imitrex, Imigran, Cinie, Illument, Migriptan), rizatriptan (Maxalt), naratriptan (Amerge, Naramig), zolmitriptan (Zomig), eletriptan (Relpax), almotriptan (Axert, Almogran), frovatriptan (Frova, Migard, Frovamig), and avitriptan (BMS-180,048).

The term “Anandamide” used hereinafter also known as N-arachidonoylethanolamine or AEA, is an endogenous cannabinoid neurotransmitter. The name is taken from the Sanskrit word (and Hinduistic religious term) ananda, which means “joy, bliss, delight”, and amide. It is synthesized from N-arachidonoyl phosphatidylethanolamine by multiple pathways. It is degraded primarily by the fatty acid amide hydrolase (FAAH) enzyme, which converts anandamide into ethanolamine and arachidonic acid. As such, inhibitors of FAAH lead to elevated anandamide levels and are being pursued for therapeutic use.

Reference is now made to pain, and more specifically headache and migraine severity scales used in the present invention to diagnose and score improvement in acute migraine attack syndromes as a result of treatment with the composition of the present invention. Examples of such scales include four poit pain severity scale, Chronic Migraine Severity Scale, Comparative Pain Scale, Wong baker scale, The Chronic Pain Index, the Migraine Disability Assessment (MIDAS), the Headache Impact Test (HIT), the Visual Analogue Scale (VAS), the 4-point verbal rating scale (VRS), the 6-point VRS, the Numerical rating scales (NRS) and the Faces Pain Rating Scale and others.

According to one embodiment, the headache pain intensity was measured on a four-point pain severity scale or the 4-point verbal rating scale (VRS), where 0=no headache; 1=mild headache; 2=moderate headache; and 3=severe headache. It is acknowledged that this scale is recommended for use in migraine research by the International Headache Society (Al-Hashel J Y, Ahmed S F, Alroughani R, Goadsby P J. Migraine among medical students in Kuwait University. The Journal of Headache and Pain 2014, 15:26; Loder E and Burch R. Measuring pain intensity in headache trials: which scale to use? Cephalalgia 2012, 32(3) 179-182).

Another example of a headache or migraine severity scale used in the present invention is the MIDAS or Migraine Disability Assessment Test, which is a test used by doctors to determine how severely migraines affect a patient's life. Patients are asked questions about the frequency and duration of their headaches, as well as how often these headaches limited their ability to participate in activities at work, at school, or at home. Once scored, the test gives the patient an idea of how debilitating his/her migraines are based on this scale: 0 to 5, MIDAS Grade I, Little or no disability; 6 to 10, MIDAS Grade II, Mild disability; 11 to 20, MIDAS Grade III, Moderate disability; and 21+, MIDAS Grade IV, Severe disability.

According to a further embodiment, the Headache Impact Test (HIT) is used to assess the effect of the cannabinoid composition of the present invention on relieving acute migraine symptoms. The HIT measures the patient's level of head pain, social, work and cognitive functioning, vitality and psychological distress. Each item is assessed a numeric value and tallied to provide an overall headache severity level. The lowest possible number is 36 and the highest is 78. Within that range there are four categories of headache severity: Little or no impact (46 or less), Some impact (50 -55), Substantial impact (56-59) and Severe impact (60-78) (i.e. http://migraine.com/blog/a-better-picture-of-migraine-related-disability-the-headache-impact-test-hit/ is incorporated herein by reference).

Another example of a migraine or headache scale included within the scope of the present invention is a scale for chronic migraine (CM), the chronic migraine severity scale (CMSS). Chronic migraine is a debilitating condition affecting 2 to 3% of the general population, and a common diagnosis in headache centers. It is one of the most difficult to treat headache disorders.

It is herein acknowledged that the scale is based on relevant aspects in the disorder. Several domains are included such as frequency, impact, analgesic consumption, psychiatric comorbidity (anxiety and mood), bodily pain and sleep quality. CMSS ranges from 0 to 100 points, 0=no impact, 100=the highest impact. CMSS includes 4 impact categories, 0-25: mild, 26-50: moderate, 51-75: severe, 75-100: very severe (Table 1). Frequency (0-30 days per month) scores one point for every headache day. Impact defined as a productivity loss higher than 50% in the day scores 0.5 for each affected day in the month. Analgesic use scores 0.5 for each day any analgesic was used. The comorbidity domain, comprising anxiety, mood-depression, bodily pain, and sleep quality each scores in a 0-10 subscale, in total 0-40. CMSS is potentially a useful clinical and research tool for evaluation and grading CM severity. It is herein noted that CMSS could also be used in other chronic daily headaches. Different treatment strategies (hospitalization, outpatient treatment, non-pharmacological treatment, polytherapy) may be indicated based on the scale severity (Mario F. P. Peres 1, Marcelo E Bigal 2; 1. IIEP, Albert Einstein Hospital, Sao Paulo, S P, Brazil; 2. Neurology, Albert Einstein College of Medicine, New York, N.Y., USA).

Reference is now made to the Pain scale which measures a patient's pain intensity or other features during the course of a treatment. Pain scales are based on self-report, observational (behavioral), and/or physiological data. Self-report is considered primary criteria for assessing the patient's feeling. Pain scales are available for neonates, infants, children, adolescents, adults, seniors, and persons whose communication is impaired, and are sometimes regarded as “the Fifth Vital Sign.”

Table 2 presents the pain scale designed for young patients:

The term “Glasgow Coma Scale” or “Glasgow Coma Score” or “GCS” refers hereinafter to a neurological scale that aims to give a reliable, objective way of recording the conscious state of a person for initial as well as subsequent assessment. A patient is assessed against the criteria of the scale, and the resulting points give a patient score between 3 (indicating deep unconsciousness) and 14 or 15. The scale is composed of three tests: eye, verbal and motor responses. The three values separately as well as their sum are considered. The lowest possible GCS (the sum) is 3 (deep coma or death), while the highest is 15 (fully awake person). GCS is used to assess level of consciousness after head injury. It is further used by first aid, EMS, nurses and doctors as being applicable to all acute medical and trauma patients. In hospitals it is also used in monitoring chronic patients in intensive care. Elements of the aforementioned scale include the following:

The term “sustained release dosage form” refers hereinafter to the release of a drug at a predetermined rate in order to maintain a constant drug concentration for a specific period of time with minimum side effects. This can be achieved through a variety of formulations, including liposomes and drug-polymer conjugates. Sustained release's definition is more akin to a “controlled release” rather than “sustained”.

According to certain aspects of the present invention, migraine is an episodic pain disorder that has the potential to develop into a chronic form, and is one of the most common neurological diseases in clinical practice, with important individual and societal implications. Migraine affects approximately 15% of the population and ranks at about number 12 among women and about 19 in the general population for the degree of disability it generates. Albeit hindered by the unpredictable, episodic nature of migraine attacks, it is noted that the migraine research i.e. provided by the present invention, greatly benefited from imaging strategies such as neuroimaging, blood flow techniques, transcranial Doppler, positron emission tomography (PET), single-photon emission tomography (SPECT) to investigate hemodynamic changes, whole-brain tractography, perfusion-weighted and diffusion-weighted imaging, implemented during and between attacks, with or without stimulation. These investigations bring an understanding of migraine as a complex sensory processing disturbance that may be associated with widespread central nervous system (CNS) dysfunction.

It is within the scope that the haemodynamic changes that take place in migraine are focused on cerebral blood flow (CBF) data measured with different techniques in patients suffering from migraine with and without aura during the different phases of attacks and in interictal periods. Special attention is paid to CBF changes detected in migraineurs as a results of the therapeutic effect of the composition of the present invention.

Reference is now made to blood oxygen level-dependent (BOLD) technique, used by the present invention as one of the techniques for assessing the effect of the composition of the present invention on relieving migraine attack symptoms. It is acknowledged that most functional magnetic resonance imaging (fMRI) studies use blood oxygen level-dependent (BOLD) contrast to measure changes in neuronal functioning associated with alterations of behavioral state. BOLD contrast reflects variation in the deoxyhemoglobin content of cerebral vessels induced by changes in the level of neuronal activity. Ultimately, deoxyhemoglobin levels depend on CMRo2, CBF, and cerebral blood volume (CBV). Thus, changes in neural activity are transduced through a hemodynamic response system to produce the observed BOLD effect.

It is further within the scope that functional magnetic resonance imaging, with the three techniques of diffusion-weighted imaging (DWI), perfusion-weighted imaging (PWI), and blood oxygenation level-dependent (BOLD) imaging, is used in the present invention to demonstrated the effect of the currently disclosed composition comprising cannabinoids on migraine attack symptoms. It is noted that fMRI is particularly useful for studying a transient phenomenon such as a migraine attack as a result of its fast time of acquisition and the possibility it offers to assess both haemodynamic and metabolic parameters. The PWI technique allows three parameters to be measured: relative CBF, relative cerebral blood volume (CBV), and mean transit time (MTT).

It is further noted that these techniques demonstrate that during visual aura, there is a reduction in CBF that is most often posterior in origin and appears to migrate anteriorly over time.

According to one embodiment, the present invention provides a composition comprising a therapeutically effective amount of Tetrahydrocannabinol (THC) or a derivative thereof, or Cannabidiol (CBD) or a derivative thereof, or a combination thereof for use in relieving migraine attack of a patient.

It is further within the scope to provide the composition as defined above, wherein the CBD and the THC are in a predefined ratio conferring relief of migraine attack symptoms.

It is further within the scope to provide the composition as defined in any of the above, wherein the CBD and the THC are in a predefined ratio conferring an additive effect with respect to relieving of migraine attack symptoms relative to the effect conferred by the CBD and the THC administered separately in a similar concentration.

It is further within the scope to provide the composition as defined in any of the above, wherein the CBD and the THC are in a predefined ratio conferring a synergistic effect with respect to relieving of migraine attack symptoms relative to the effect conferred by the CBD and the THC administered separately in a similar concentration.

It is further within the scope to provide the composition as defined in any of the above, wherein the predefined ratio of the CBD and the THC is selected from the group consisting of about 1:1, 1:5, 5:1, 1:4 and 4:1, respectively.

It is further within the scope to provide the composition as defined in any of the above, wherein the symptoms are selected from the group consisting of nausea, vomiting, sensitivity to light, sensitivity to sound, sensitivity to smell and any combination thereof.

It is further within the scope to provide the composition as defined in any of the above, wherein the THC or a derivative thereof is selected from the group consisting of THC, THCV, THCA, THCVA and any combination thereof.

It is further within the scope to provide the composition as defined in any of the above, wherein the THC or a derivative thereof is selected from the group consisting of natural THC or a derivative thereof produced in the body of humans and animals, THC or a derivative thereof extracted from plants, synthetic THC or a derivative thereof, and any combination thereof.

It is further within the scope to provide the composition as defined in any of the above, wherein the THC or a derivative thereof is extracted from cannabis plant; the cannabis plant is selected from a group consisting of: Cannabis sativa, Cannabis indica, Cannabis ruderalis, and any combination thereof.

It is further within the scope to provide the composition as defined in any of the above, wherein the composition provides a synergistic effect with respect to relieving acute migraine attack as compared to the effect provided by THC or a derivative thereof or by CBD or a derivative thereof administered separately in a similar concentration.

It is further within the scope to provide the composition as defined in any of the above, wherein the Cannabidiol (CBD) or a derivative thereof is selected from the group consisting of CBD, CBDV, CBDA and any combination thereof.

It is further within the scope to provide the composition as defined in any of the above, wherein the CBD or a derivative thereof is selected from the group consisting of natural CBD or a derivative thereof produced in the body of humans and animals, CBD or a derivative thereof extracted from plants, synthetic CBD or a derivative thereof, and any combination thereof.

It is further within the scope to provide the composition as defined in any of the above, wherein the CBD or a derivative thereof is extracted from cannabis plant; the cannabis plant is selected from a group consisting of: Cannabis sativa, Cannabis indica, Cannabis ruderalis, and any combination thereof.

It is further within the scope to provide the composition as defined in any of the above, wherein the concentration of the THC or a derivative thereof in the composition is in the range of about 0.2% to about 10%.

It is further within the scope to provide the composition as defined in any of the above, wherein the composition is configured for administration in a dosage unit of between about 0.5 mg to about 50 mg of the THC or a derivative thereof.

It is further within the scope to provide the composition as defined in any of the above, wherein the composition comprises between about 1 wt. % to about 10 wt. % of the THC or a derivative thereof.

It is further within the scope to provide the composition as defined in any of the above, wherein the concentration of the CBD or a derivative thereof in the composition is in the range of about 1 to about 50%.

It is further within the scope to provide the composition as defined in any of the above, wherein the composition is configured for administration in a dosage unit of between about 1 mg to about 300 mg of the CBD or a derivative thereof.

It is further within the scope to provide the composition as defined in any of the above, wherein the composition comprises between about 1 wt. % to about 30 wt. % of the CBD or a derivative thereof.

It is further within the scope to provide the composition as defined in any of the above, wherein the concentration of the CBD or the derivative thereof is in the range of about 2% (wt.) to about 20%. (wt.).

It is further within the scope to provide the composition as defined in any of the above, wherein the concentration of the THC or the derivative thereof is in the range of about 2% (wt.) to about 20% (wt.).

It is further within the scope to provide the composition as defined in any of the above, wherein the composition comprises cannabis oil.

It is further within the scope to provide the composition as defined in any of the above, wherein the cannabis oil is in a concentration of about 2% (wt.) to about 25% (wt.).

It is further within the scope to provide the composition as defined in any of the above, wherein the migraine attack is selected from the group consisting of acute migraine attack and chronic migraine attack.

It is further within the scope to provide the composition as defined in any of the above, wherein the composition provides an improvement in migraine attack symptoms of the patient measured by at least one pain severity scale, compared to an established baseline or placebo.

It is further within the scope to provide the composition as defined in any of the above, wherein the improvement in migraine attack symptoms of the patient is measured by a decrease in the patient's score of at least one point or level on the at least one pain severity scale, as compared to an established baseline or to a placebo.

It is further within the scope to provide the composition as defined in any of the above, wherein the improvement in migraine attack symptoms of the patient is measured by a decrease in the patient's score of at least two points at the first two hours from the administration of the composition to the patient, on the at least one pain severity scale, as compared to an established baseline or to a placebo.

It is further within the scope to provide the composition as defined in any of the above, wherein the pain severity scale is selected from a group consisting of four point pain severity scale, Chronic Migraine Severity Scale, Comparative Pain Scale, Wong baker scale, The Chronic Pain Index, the Migraine Disability Assessment (MIDAS), the Headache Impact Test (HIT), the Visual Analogue Scale (VAS), the 4-point verbal rating scale (VRS), the 6-point VRS, the Numerical rating scales (NRS), the Faces Pain Rating Scale and any combination thereof.

It is further within the scope to provide the composition as defined in any of the above, wherein the symptoms are selected from a group consisting of nausea, vomiting, sensitivity to light, sensitivity to sound, sensitivity to smell and any combination thereof.

It is further within the scope to provide the composition as defined in any of the above, wherein the composition is formulated for an administration route selected from the group consisting of: intranasal, transdermal, intravenous, vaginal, sublingual, buccal, oral, and any combination thereof.

It is further within the scope to provide the composition as defined in any of the above, wherein the composition is formulated in a dosage form selected from the group consisting of liquid, solid, gas, oral, sublingual, pill, tablet, capsule, buccal, tincture, strip, film, spray, lozenge, effervescent form, sub-lingual, granules, orally-disintegrating, thin film, liquid, solution, suspension, emulsion, powder or liquid or solid crystals, powder, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, syrup, skin patch, vaginal, suppository, pessary, rectal and any combination thereof.

It is further within the scope to provide the composition as defined in any of the above, wherein the composition is administered in combination with at least one migraine therapeutic agent.

It is further within the scope to provide the composition as defined in any of the above, wherein the at least one migraine therapeutic agent is selected from a group consisting of pain-relieving medications, aspirin, acetaminophen, indomethacin, Ergots, anti-nausea medications, NSAID, triptan, Opioid medications, Glucocorticoids such as prednisone and dexamethasone, Cardiovascular drugs, Antidepressants, Anti-seizure drugs, OnabotulinumtoxinA (Botox), Pain relievers and any combination thereof.

It is further within the scope to provide the composition as defined in any of the above, wherein the composition combined with at least one migraine therapeutic agent provides a synergistic effect with respect to relieving migraine attack symptoms relative to the effect provided by the migraine therapeutic agent administered separately.

It is further within the scope to provide the composition as defined in any of the above, wherein the composition additionally comprises at least one carrier or excipient selected from the group consisting of diluent, solvent, absorbent, anti-adherent, binder, coatings, disintegrant, surfactant, dissolving agent, solubilizing agent, bioadhesive agent, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents, glidant, flavours, colours, sweetener, thickener, lubricant, sorbents, preservatives, and any combination thereof.

It is further within the scope to provide the composition as defined in any of the above, wherein the solvent is ethanol.

It is further within the scope to provide the composition as defined in any of the above, wherein the composition is substantially free of a pharmaceutically acceptable emulsifying agent or surfactant.

It is further within the scope to provide the composition as defined in any of the above, wherein the composition is formulated in a sublingual dosage form.

It is further within the scope to provide the composition as defined in any of the above, wherein the composition is formulated in a solid dosage form.

It is further within the scope to provide the composition as defined in any of the above, wherein the THC and the CBD are formulated for penetrating the mucosal barrier.

It is further within the scope to provide the composition as defined in any of the above, wherein the composition is formulated for rapid disintegration upon administration.

It is further within the scope to provide the composition as defined in any of the above, wherein the composition is not significantly psychoactive.

It is further within the scope to provide the composition as defined in any of the above, wherein the composition is in a sustained release dosage form or in an immediate release dosage form.

It is further within the scope to provide the composition as defined in any of the above, wherein the sustained release dosage form is selected from the group consisting of liposomes, drug polymer conjugates, microencapsulation, controlled-release tablet coating, and any combination thereof.

It is further within the scope to provide the composition as defined in any of the above, wherein the composition is in an effervescent form.

It is further within the scope to provide the composition as defined in any of the above, wherein the composition is administered once, twice, three or four times through the day.

It is further within the scope to provide the composition as defined in any of the above, wherein administration of the composition to a patient suffering from migraine attack increases cerebral blood flow (CBF) as measure by functional magnetic resonance imaging (fMRI), in comparison with the cerebral blood flow (CBF) of the patient prior to the administration of the composition to the patient or as compared to a healthy control.

It is further within the scope to provide the composition as defined in any of the above, wherein the fMRI is selected from the group consisting of: diffusion-weighted imaging (DWI), perfusion-weighted imaging (PWI), blood oxygenation level-dependent (BOLD) imaging, and any combination thereof.

It is further within the scope to provide the composition as defined in any of the above, wherein administration of the composition to a patient suffering from migraine attack, reduces paroxysmal cortical neuron depolarization as measure by electroencephalogram (EEG) or functional magnetic resonance imaging (fMRI), in comparison with the paroxysmal cortical neuron depolarization of the patient prior to the administration of the composition to the patient or as compared to a healthy control.

It is further within the scope to provide the composition as defined in any of the above, wherein the composition is dissolved in a lipophilic solvent or suspension carrier.

It is further within the scope to provide the composition as defined in any of the above, wherein the lipophilic solvent or suspension carrier are selected from a group consisting of ethanol, medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

It is further within the scope to provide a synergistically effective composition, wherein the composition comprising a therapeutically effective amount of Cannabidiol (CBD) or a derivative thereof and Tetrahydrocannabinol (THC) or a derivative thereof in a predefined ratio conferring a synergistic effect with respect to relieving migraine attack of a patient, relative to the effect of the CBD and the THC administered separately in a similar concentration.

It is further within the scope to provide the synergistically effective composition as defined above, wherein the predefined ratio of the CBD and the THC is selected from the group consisting of: about 1:1, 5:1, 1:5, 1:4 and 4:1 respectively.

It is a further embodiment of the present invention to provide a method for relieving migraine attack of a patient comprising steps of: (a) providing a composition comprising Tetrahydrocannabinol (THC) or a derivative thereof, or Cannabidiol (CBD) or a derivative thereof, or a combination thereof; and (b) administering the composition to the patient at a therapeutically effective dosage for relieving migraine attack of the patient.

It is further within the scope of the present invention to provide the method as defined above, additionally comprising steps of providing the composition comprising the CBD and the THC in a predefined ratio conferring relief of migraine attack symptoms.

It is further within the scope of the present invention to provide the method as defined in any of the above, wherein the CBD and the THC are in a predefined ratio conferring an additive effect with respect to relieving of migraine attack symptoms relative to the effect conferred by the CBD and the THC administered separately in a similar concentration.

It is further within the scope of the present invention to provide the method as defined in any of the above, wherein the CBD and the THC are in a predefined ratio conferring a synergistic effect with respect to relieving of migraine attack symptoms relative to the effect conferred by the CBD and the THC administered separately in a similar concentration.

It is further within the scope of the present invention to provide the method as defined in any of the above, additionally comprising steps of selecting the predefined ratio of the CBD and the THC from the group consisting of about 1:1, 1:5, 5:1, 1:4 and 4:1, respectively.

It is further within the scope of the present invention to provide the method as defined in any of the above, additionally comprising steps of selecting the symptoms from the group consisting of nausea, vomiting, sensitivity to light, sensitivity to sound, sensitivity to smell and any combination thereof.

It is further within the scope of the present invention to provide the method as defined in any of the above, additionally comprising steps of selecting the THC or a derivative thereof from the group consisting of THC, THCV, THCA, THCVA and any combination thereof.

It is further within the scope of the present invention to provide the method as defined in any of the above, additionally comprising steps of selecting the THC or a derivative thereof from the group consisting of natural THC or a derivative thereof produced in the body of humans and animals, THC or a derivative thereof extracted from plants, synthetic THC or a derivative thereof, and any combination thereof.

It is further within the scope of the present invention to provide the method as defined in any of the above, additionally comprising steps of extracting the THC or a derivative thereof from cannabis; the cannabis is selected from a group consisting of: Cannabis sativa, Cannabis indica, Cannabis ruderalis, and any combination thereof.

It is further within the scope of the present invention to provide the method as defined in any of the above, additionally comprising steps of providing a synergistic effect with respect to relieving acute migraine attack as compared to the effect provided by the THC or a derivative thereof or by the CBD or a derivative thereof administered separately in a similar concentration.

It is further within the scope of the present invention to provide the method as defined in any of the above, additionally comprising steps of selecting the Cannabidiol (CBD) or a derivative thereof from the group consisting of CBD, CBDV, CBDA and any combination thereof.

It is further within the scope of the present invention to provide the method as defined in any of the above, additionally comprising steps of selecting the CBD or a derivative thereof from the group consisting of natural CBD or a derivative thereof produced in the body of humans and animals, CBD or a derivative thereof extracted from plants, synthetic CBD or a derivative thereof, and any combination thereof.

It is further within the scope of the present invention to provide the method as defined in any of the above, additionally comprising steps of extracting the CBD or a derivative thereof from cannabis; the cannabis is selected from a group consisting of: Cannabis sativa, Cannabis indica, Cannabis ruderalis, and any combination thereof.

It is further within the scope of the present invention to provide the method as defined in any of the above, additionally comprising steps of providing the THC or a derivative thereof in a concentration in the range of about 0.2% to about 10%.

It is further within the scope of the present invention to provide the method as defined in any of the above, additionally comprising steps of providing the composition configured for administration in a dosage unit of between about 0.5 mg to about 50 mg of the THC or a derivative thereof.

It is further within the scope of the present invention to provide the method as defined in any of the above, additionally comprising steps of formulating the composition comprising between about 1 wt. % to about 10 wt. % of the THC or a derivative thereof.

It is further within the scope of the present invention to provide the method as defined in any of the above, additionally comprising steps of formulating the composition comprising CBD or a derivative thereof concentration in the range of about 1% to about 50%.

It is further within the scope of the present invention to provide the method as defined in any of the above, additionally comprising steps of formulating the composition configured for administration in a dosage unit of between about 1 mg to about 300 mg of the CBD or a derivative thereof.

It is further within the scope of the present invention to provide the method as defined in any of the above, additionally comprising steps of formulating the composition comprising between about 1 wt. % to about 30 wt. % of the CBD or a derivative thereof.

It is further within the scope of the present invention to provide the method as defined in any of the above, wherein the concentration of the CBD or the derivative thereof is in the range of about 2% (wt.) to about 20%. (wt.).

It is further within the scope of the present invention to provide the method as defined in any of the above, wherein the concentration of the THC or the derivative thereof is in the range of about 2% (wt.) to about 20% (wt.).

It is further within the scope of the present invention to provide the method as defined in any of the above, wherein the composition comprises cannabis oil.

It is further within the scope of the present invention to provide the method as defined in any of the above, wherein the cannabis oil is in a concentration of about 2% (wt.) to about 25% (wt.).

It is further within the scope of the present invention to provide the method as defined in any of the above, additionally comprising steps of selecting the migraine attack from the group consisting of acute migraine attack and chronic migraine attack.

It is further within the scope of the present invention to provide the method as defined in any of the above, additionally comprising steps of administering the composition to the patient at a therapeutically effective dosage so as to provide an improvement in migraine attack symptoms of the patient as measured by at least one pain severity scale, compared to an established baseline or placebo.

It is further within the scope of the present invention to provide the method as defined in any of the above, additionally comprising steps of administering the composition to the patient at a therapeutically effective dosage so as to provide an improvement in migraine attack symptoms of the patient as measured by a decrease in the patient's score of at least one point or level on the at least one pain severity scale, as compared to an established baseline or to a placebo.

It is further within the scope of the present invention to provide the method as defined in any of the above, additionally comprising steps of administering the composition to the patient at a therapeutically effective dosage so as to provide an improvement in migraine attack symptoms of the patient as measured by a decrease in the patient's score of at least two points or levels at the first two hours from administration of the composition to the patient, on the at least one pain severity scale, as compared to an established baseline or to a placebo.

It is further within the scope of the present invention to provide the method as defined in any of the above, additionally comprising steps of selecting the pain severity scale from a group consisting of four point pain severity scale, Chronic Migraine Severity Scale, Comparative Pain Scale, Wong baker scale, The Chronic Pain Index, the Migraine Disability Assessment (MIDAS), the Headache Impact Test (HIT), the Visual Analogue Scale (VAS), the 4-point verbal rating scale (VRS), the 6-point VRS, the Numerical rating scales (NRS) and the Faces Pain Rating Scale and any combination thereof.

It is further within the scope of the present invention to provide the method as defined in any of the above, additionally comprising steps of selecting the symptoms from the group consisting of nausea, vomiting, sensitivity to light, sensitivity to sound, sensitivity to smell and any combination thereof.

It is further within the scope of the present invention to provide the method as defined in any of the above, additionally comprising steps of formulating the composition for an administration route selected from the group consisting of: intranasal, transdermal, intravenous, vaginal, sublingual, buccal, oral, and any combination thereof.

It is further within the scope of the present invention to provide the method as defined in any of the above, additionally comprising steps of formulating the composition in a dosage form selected from the group consisting of liquid, solid, gas, oral, sublingual, pill, tablet, capsule, buccal, tincture, strip, film, spray, lozenge, effervescent form, sub-lingual, granules, orally-disintegrating, thin film, liquid, solution, suspension, emulsion, powder or liquid or solid crystals, powder, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, syrup, skin patch, vaginal, suppository, pessary, rectal and any combination thereof.

It is further within the scope of the present invention to provide the method as defined in any of the above, additionally comprising steps of administering the composition in combination with at least one migraine therapeutic agent.

It is further within the scope of the present invention to provide the method as defined in any of the above, additionally comprising steps of selecting the at least one migraine therapeutic agent from a group consisting of pain-relieving medications, aspirin, acetaminophen, indomethacin, Ergots, anti-nausea medications, NSAID, triptan, Opioid medications, Glucocorticoids such as prednisone and dexamethasone, Cardiovascular drugs, Antidepressants, Anti-seizure drugs, OnabotulinumtoxinA (Botox), Pain relievers and any combination thereof.

It is further within the scope of the present invention to provide the method as defined in any of the above, additionally comprising steps of providing a synergistic effect with respect to relieving migraine attack symptoms relative to the effect provided by the migraine therapeutic agent administered separately in a similar concentration.

It is further within the scope of the present invention to provide the method as defined in any of the above, additionally comprising steps of formulating the composition with at least one carrier or excipient selected from the group consisting of diluent, solvent, absorbent, anti-adherent, binder, coatings, disintegrant, surfactant, dissolving agent, solubilizing agent, bioadhesive agent, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents, glidant, flavours, colours, sweetener, thickener, lubricant, sorbents, preservatives, and any combination thereof.

It is further within the scope of the present invention to provide the method as defined in any of the above, wherein the solvent is ethanol.

It is further within the scope of the present invention to provide the method as defined in any of the above, additionally comprising steps of formulating the composition substantially free of a pharmaceutically acceptable emulsifying agent or surfactant.

It is further within the scope of the present invention to provide the method as defined in any of the above, additionally comprising steps of formulating the composition in a sublingual dosage form.

It is further within the scope of the present invention to provide the method as defined in any of the above, additionally comprising steps of formulating the composition in a solid dosage form.

It is further within the scope of the present invention to provide the method as defined in any of the above, additionally comprising steps of formulating the composition for penetrating the mucosal barrier.

It is further within the scope of the present invention to provide the method as defined in any of the above, additionally comprising steps of formulating the composition for rapid disintegration upon administration.

It is further within the scope of the present invention to provide the method as defined in any of the above, wherein the composition is not significantly psychoactive.

It is further within the scope of the present invention to provide the method as defined in any of the above, additionally comprising steps of formulating the composition in a sustained release dosage form or in an immediate release dosage form.

It is further within the scope of the present invention to provide the method as defined in any of the above, additionally comprising steps of selecting the sustained release dosage form from a group consisting of liposomes, drug polymer conjugates, microencapsulation, controlled-release tablet coating, and any combination thereof.

It is further within the scope of the present invention to provide the method as defined in any of the above, additionally comprising steps of administering the composition once, twice, three or four times through the day.

It is a further embodiment of the present invention to provide a method for increasing cerebral blood flow (CBF) in a patient suffering from migraine attack, the method comprises steps of: (a) administering a composition comprising Tetrahydrocannabinol (THC) or a derivative thereof, or Cannabidiol (CBD) or a derivative thereof, or a combination thereof to the patient at a therapeutically effective dosage; and (b) detecting increase in cerebral blood flow (CBF) of the patient by functional magnetic resonance imaging (fMRI) analysis.

It is further within the scope of the present invention to provide the method as defined in any of the above, additionally comprises steps of selecting the fMRI from the group consisting of: diffusion-weighted imaging (DWI), perfusion-weighted imaging (PWI), blood oxygenation level-dependent (BOLD) imaging, and any combination thereof.

It is further within the scope of the present invention to provide a method for reducing paroxysmal cortical neuron depolarization in a patient suffering from migraine attack comprising steps of: (a) administering to the patient a composition comprising Tetrahydrocannabinol (THC) or a derivative thereof, or Cannabidiol (CBD) or a derivative thereof, or a combination thereof at a therapeutically effective dosage; and (b) detecting decrease in paroxysmal cortical neuron depolarization of the patient by electroencephalogram (EEG) or functional magnetic resonance imaging (fMRI) analysis.

It is a further embodiment of the present invention to disclose a use of a composition comprising a therapeutically effective amount of Tetrahydrocannabinol (THC) or a derivative thereof, or Cannabidiol (CBD) or a derivative thereof, or a combination thereof in the manufacture of a medicament for relieving migraine attack of a patient.

It is further within the scope of the present invention to disclose the use as defined above, wherein the Tetrahydrocannabinol (THC) or a derivative thereof, or Cannabidiol (CBD) or a derivative thereof, or the combination thereof, is extracted from cannabis plant.

It is a further embodiment of the present invention to provide a composition comprising a therapeutically effective amount of Cannabidiol (CBD) or a derivative thereof or a Tetrahydrocannabinol (THC) or a derivative thereof for use in relieving migraine attack of a patient, wherein the composition is prepared by steps of: (a) preparing a mixture comprising an effective amount of cannabis oil, by a wet granulation process; and, (b) formulating the mixture in a solid dosage form by direct compression.

It is further within the scope of the present invention to provide the composition prepared by steps as defined above, wherein the mixture is further prepared by steps of: (a) preparing a first mixture comprising the cannabis oil and a solvent; (b) preparing a second mixture comprising at least one pharmaceutically acceptable carrier or excipient selected from the group consisting of a sweetener, a disintegrant, a thickener and any combination thereof; and (c) adding the second mixture to the first mixture by mixing using a high shear granulator.

It is further within the scope of the present invention to provide the composition prepared by steps as defined in any of the above, wherein the composition is further prepared by steps of: preparing the first mixture comprising cannabis oil, absorbent, lubricant and binder.

It is further within the scope of the present invention to provide the composition prepared by steps as defined in any of the above, wherein the composition is further prepared by steps of: (a) drying the mixture of step c to LOD equal or less than 1%; and (b) mixing the dried mixture with at least one pharmaceutically acceptable carrier or excipient selected from the group consisting of: glidant, binder, sweetener, lubricant, disintegrant and any combination thereof.

According to a further embodiment, the present invention provides a composition comprising Tetrahydrocannabinol (THC) or a derivative thereof, or Cannabidiol (CBD) or a derivative thereof, or a combination thereof wherein administration of the composition to a patient suffering from migraine attack increases cerebral blood flow (CBF) as measure by functional magnetic resonance imaging (fMRI), in comparison with the cerebral blood flow (CBF) of the patient prior to the administration of the composition to the patient or as compared to a healthy control.

It is further within the scope to provide the composition as defined in any of the above, wherein the fMRI is selected from the group consisting of: diffusion-weighted imaging (DWI), perfusion-weighted imaging (PWI), blood oxygenation level-dependent (BOLD) imaging, and any combination thereof.

According to a further embodiment, the present invention provides a composition comprising Tetrahydrocannabinol (THC) or a derivative thereof, or Cannabidiol (CBD) or a derivative thereof, or a combination thereof, wherein administration of the composition to a patient suffering from migraine attack, reduces paroxysmal cortical neuron depolarization as measure by electroencephalogram (EEG) or functional magnetic resonance imaging (fMRI), in comparison with the paroxysmal cortical neuron depolarization of the patient prior to the administration of the composition to the patient or as compared to a healthy control.

In order to understand the invention and to see how it may be implemented in practice, a plurality of preferred embodiments will now be described, by way of non-limiting example only, with reference to the following examples.

### EXAMPLE 1

### A Protocol for a Clinical Trial Assessing the Effect of the Composition of the Present Invention Comprising THC, CBD or Derivatives or Combinations Thereof on Reliving Acute Migraine Attack

### Objectives:

The effect of Tetrahydrocannabinol (THC), Cannabidiol (CBD) and derivatives and combinations thereof vs. NSAIDs (such as ibuprofen), was assessed in relieving acute migraine attack.

### Study Design:

This example provides a prospective randomized double-blind, placebo-controlled trial.

The study population includes patients presenting with chief complaint an acute migraine attack to the emergency department (ED) at Soroka University Medical Center.

The study population is randomized into “double dummy” design (ratio 1:1):
Group 1—received drops comprising at least one of the following cannabinoids: Tetrahydrocannabinol (THC), Cannabidiol (CBD) or a combination thereof, and placebo tab ibuprofen.
Group 2—received placebo drops comprising Tetrahydrocannabinol (THC) or Cannabidiol (CBD) or a combination thereof, and tab ibuprofen.

It is noted that the composition tested in this study comprises at least one of following cannabinoids or combinations of cannabinoids dosages: 1) THC at a dosage range of about 10 mg to about 50 mg; 2) CBD at about 50 mg; 3) THC at about 10 mg and CBD at about 5 mg; 4) THC and CBD at equal dosages of 20 mg; and 5) CBD at about 50 mg and THC at about 5 mg.

Headache and nausea severity is assessed using a pain or migraine or headache severity scale, preferably, the 4-point pain severity scale, which was applied before treatment, every 15 minutes during the first two hours of treatment, and every hour afterwards until discharge. Additionally, THC and CBD blood levels are measured at about 30, 60 minutes and 2 hours. Patients are observed at ED for at least 4 hours and transportation home is arranged (Ferrari MD1, Goadsby P J, Roon K I, Lipton R B.Triptans (serotonin, 5-HT1B/1D agonists) in migraine: detailed results and methods of a meta-analysis of 53 trials. Cephalalgia. 2002 Oct;22(8):633-58).

At 24h from ED admission, patient is interviewed again for the patient global assessment (PGA) and estimates of attack severity, treatment satisfaction and incidence of adverse events.

### Study Population:

The study population includes patients presenting with chief complaint of acute migraine attack of moderate to severe intensity to the ED at Soroka University Medical Center diagnosed by a board certified neurologist during the ED stay.

### Primary Endpoint:

Primary endpoint is treatment failure defined as one of the following:
No improvement in migraine intensity from baseline during the first hour after treatment defined as a decrease of one level in pain intensity by the 4-point pain severity scale.
No sufficient improvement (at least 2 levels) in migraine severity (headache) from baseline during at least two hours after the treatment initiation.

### Sample Size Considerations:

The Primary hypothesis is:

The treatment failure rate will be significantly lower using THC and/or CBD vs. NSAIDS.

H0: π2=π1

H1: π2≠π1

Where π1 is the rate of the primary endpoint (treatment failure rate) in group 1 and n2 in group 2. According to the previous studies, NSAIDS treatment failure rate is expected to be 70%. The expected THC and/or CBD treatment failure rate is 40%. With alpha (two-sided) <0.05 and 80% power, each arm should include 41 patients. The sample size is increased to 45 subjects in each arm to allow for attrition.

Secondary Outcomes and Analyses:

Safety endpoint:

Composite endpoint of serious adverse events (SAE)

Other
1. Patient general assessment at discharge and at 24 h.
2. Overall satisfaction level at discharge and at 24 h
3. Nausea level over ED stay assessed every 15 minutes during the first two hours, and every hour afterwards until discharge.
4. Pain level over ED stay assessed every 15 minutes during the first two hours, and every hour afterwards until discharge.
5. Subgroup analyses by gender, age and baseline migraine severity.

### Inclusion Criteria:

1. Males and females >18 years old
2. Previously diagnosed as suffering from Migraine with or without aura, according to the ICHD-III (34)
3. Admitted to ED at Soroka University Medical Center.
4. Diagnosed with acute migraine attack with severity level above 1 on the 4-point pain severity scale.
5. Signed informed consent
6. Agreement to avoid driving for at least 10 hours following the treatment.

### Exclusion Criteria:

1. Confusion state or Glasgow Coma Score<15
2. History of substance abuse
3. History of psychiatric disorder
4. Ongoing medical THC or CBD use
5. Narrow angle glaucoma
6. Peptic disease
7. Pregnancy

### Rescue Management:

Treatment failure defined as no improvement in a pain or headache or migraine severity scale, e.g. 4-point pain severity scale at the first hour, and less than two levels improvement at 2 hours from the baseline assessment. This will trigger rescue treatment using parenteral NSAID or triptan, according to the standard clinical practice.

### Data Management and Analysis:

The study is overseen and managed by the Soroka Clinical Research Center.

### Results:

It appears from the results of this study that the cannabinoid composition of the present invention comprising THC or CBD or a combination thereof, is more efficacious in providing an improvement in migraine symptoms as compared to the effect provided by NSAID agent such as ibuprofen, or triptan agent, administered according to the standard clinical practice. This improvement in the headache and nausea severity symptoms provided by the composition of the present invention is demonstrated by a decrease in the score of a patient suffering from a migraine attack on at least one recognized pain or headache or migraine severity scale such as the 4-point pain severity scale.

It further appears from the results of this study that a synergistic effect with respect to relieving migraine attack symptoms is provided when administering to a patient suffering from a migraine attack, the cannabinoid composition of the present invention in combination with NSAID or any other conventional migraine therapeutic agent.

It is noted that Cannabinol (CBN), Cannabichromene (CBC), the acids (CBDA, CBGA, THCA) and propyl homologues (CBDV, CBGV, THCV) of CBD, Cannabigerol (CBG) and THC, and Tetrahydrocannabivarin acid (THCVA) were also tested by the above or similar protocol, with required minor modifications.

### EXAMPLE 2

### A protocol for a Clinical Trial Assessing the Effect of THC or a Derivative Thereof on Reliving Acute Migraine Attack

### Objectives:

To assess the effect of Tetrahydrocannabinol (THC) or a derivative thereof vs. NSAIDs (such as ibuprofen) in relieving acute migraine attack.

### Study Design:

This example provides a prospective randomized double-blind, placebo-controlled trial.

The study population includes patients presenting with chief complaint an acute migraine attack to the emergency department (ED) at Soroka University Medical Center.

The study population is randomized into “double dummy” design (ratio 1:1):
Group 1—received Tetrahydrocannabinol (THC) and placebo tab ibuprofen.
Group 2—received placebo Tetrahydrocannabinol (THC) drops and tab ibuprofen.

The THC study doses include dose rages of between about 5 mg and up to 50 mg THC, preferably dose ranges of between about 10 mg THC and about 30 mg THC.

According to one embodiment, an ethanol extracted cannabis oil dissolved in a vegetable oil (with a distinct taste to differentiate it from the placebo oil) is used in this study. The aforementioned cannabis composition comprises THC dose range of about 20 mg to about 40 mg.

Headache and nausea severity is assessed using a 4-point pain severity scale (score 0-3) as detailed above. The pain severity scale was applied to the study population before treatment, every 15 minutes during the first two hours of treatment, and every hour afterwards until discharge. Additionally, THC blood levels are measured at about 30, 60 minutes and 2 hours. Patients are observed at ED for at least 4 hours and transportation home is arranged (Ferrari MD1, Goadsby P J, Roon K I, Lipton R B. Triptans (serotonin, 5-HT1B/1D agonists) in migraine: detailed results and methods of a meta-analysis of 53 trials. Cephalalgia. 2002 October; 22(8):633-58).

At 24 h from ED admission, patient is interviewed again for the patient global assessment (PGA) and estimates of attack severity, treatment satisfaction and incidence of adverse events.

### Study Population:

The study population includes patients presenting with chief complaint of acute migraine attack of moderate to severe intensity to the ED at Soroka University Medical Center diagnosed by a board certified neurologist during the ED stay.

### Primary Endpoint:

Primary endpoint is treatment failure defined as one of the following:
No improvement in migraine intensity from baseline during the first hour after treatment defined as a decrease of one level in pain intensity by the 4-point pain severity scale.
No sufficient improvement (at least 2 levels) in migraine severity (headache) from baseline during at least two hours after the treatment initiation.

### Sample Size Considerations:

The Primary hypothesis is:

The treatment failure rate will be significantly lower using THC vs. NSAIDS.

H0: π2=π1

H1: π2≠π1

Where π1 is the rate of the primary endpoint (treatment failure rate) in group 1 and n2 in group 2. According to the previous studies, NSAIDS treatment failure rate is expected to be 70%. The expected THC treatment failure rate is 40%. With alpha (two-sided) <0.05 and 80% power, each arm should include 41 patients. The sample size is increased to 45 subjects in each arm to allow for attrition.

### Secondary Outcomes and Analyses:

Safety endpoint:

Composite endpoint of serious adverse events (SAE)

Other
1. Patient general assessment at discharge and at 24 h.
2. Overall satisfaction level at discharge and at 24 h
3. Nausea level over ED stay assessed every 15 minutes during the first two hours, and every hour afterwards until discharge.
4. Pain level over ED stay assessed every 15 minutes during the first two hours, and every hour afterwards until discharge.
5. Subgroup analyses by gender, age and baseline migraine severity.

### Inclusion Criteria:

1. Males and females >18 years old
2. Previously diagnosed as suffering from Migraine with or without aura, according to the ICHD-III (34)
3. Admitted to ED at Soroka University Medical Center.
4. Diagnosed with acute migraine attack with severity level above 1 on 4-point pain severity scale.
5. Signed informed consent
6. Agreement to avoid driving for at least 10 hours following the treatment.

### Exclusion Criteria:

1. Confusion state or Glasgow Coma Score<15
2. History of substance abuse
3. History of psychiatric disorder
4. Ongoing medical THC use
5. Narrow angle glaucoma
6. Peptic disease
7. Pregnancy

### Rescue Management:

Treatment failure defined as no improvement in a pain scale, such as the 4-point pain severity scale, at the first hour and less than two levels improvement at 2 hours from the baseline assessment. This will trigger rescue treatment using parenteral NSAID or triptan, according to the standard clinical practice.

### Data Management and Analysis:

The study is overseen and managed by the Soroka Clinical Research Center.

### Results:

It appears from the results of this study that the composition comprising THC of the present invention is effective in treatment of acute migraine attack. More specifically, it is shown to be more efficacious in relieving acute migraine attack symptoms as compared to the effect provided by NSAID or triptan agent, administered according to the standard clinical practice. This improvement in headache and nausea severity symptoms provided by the composition of the present invention is demonstrated by a decrease in the score of patients suffering from a migraine attack as measured by at least one pain severity scale.

Moreover, it appears from the results of this study that a synergistic effect with respect to relieving acute migraine attack symptoms is provided when administering the cannabinoid composition of the present invention in combination with NSAID or any other conventional migraine therapeutic agent, to a patient suffering from a migraine attack.

It is noted that Cannabinol (CBN), Cannabichromene (CBC), the acids (CBDA, CBGA, THCA) and propyl homologues (CBDV, CBGV, THCV) of CBD, Cannabigerol (CBG) and THC, and Tetrahydrocannabivarin acid (THCVA) were also tested by the above or similar protocol, with required minor modifications.

### EXAMPLE 3

### A Solid Formulation Containing Cannabinoid(s)

Reference is now made to a solid formulation with enhanced penetration of cannabinoid (i.e. THC and/or CBD) through oral administration. Using a wet granulation technology, cannabis oil is combined with dry powder components to produce a tablet with good hardness characteristics which disintegrates rapidly upon administration. The THC and/or CBD ingredients in the resultant tablet or other solid form can penetrate the mucosal barrier without emulsification.

Reference is now made to Table 3 presenting ingredients and production process of a solid oral formulation containing cannabis oil to provide 10 mg of THC and 2.5 mg of CBD (40% of THC and 10% of CBD), as an embodiment of the present invention.

TABLE 3
A solid formulation containing cannabis oil

| Core | | | | |
|---|---|---|---|---|
| %/ | mg/ | Liquids | | |
| tablet | tablet | mg/tablet | Raw Materials | Function |
| | | | PART I Wet Granulation | |
| 12.5 | 25.00 | | Cannabis Oil | API |
| | | 20.0 | Ethanol | Solvent |
| 20 | 40.00 | | Mannitol | Sweetener |
| | | | | disintegrant |
| 12.5 | 25.00 | | Plasdone K25 | Thickener |
| | | | PART II Direct Compression | |
| 20.5 | 41.00 | | Corn Starch | Disintegrant |
| | | | | binder |
| 25 | 50.00 | | Mannitol | Sweetener |
| | | | | disintegrant |
| 1.5 | 3.00 | | Magnesium Stearate | Lubricant |
| 5.0 | 10.00 | | Aerosil 200 | Glidant |
| | | | (Silicone Dioxide) | |
| 3.0 | 6.00 | | Croscarmellose Sodium | Disintegrant |
| 100 | 200 | | Total | |

Reference is now made to manufacturing steps of the solid formulation comprising cannabis oil:
1. Slowly adding Ethanol to Cannabis oil throughout an intensive mixing and observing liquid homogeneity. A
2. Mixing Mannitol and Plasdone 25 (Polymer of 1-vinyl-2-pyrrolidone) in a blender. B
3. Slowly adding B to A with mixing (use high-shear granulator). C
4. Drying C at 80° C. until LOD less than 1%. D
5. Milling D and sieving with 120 micron screen sieve. E
6. Mixing in a blender E with Corn Starch, Mannitol, Magnesium Stearate, Silica and Croscarmelose Sodium. F
7. Compressing tablets with a tableting press machine.

It is within the scope that a solid formulation containing cannabis oil as described above has therapeutic effect on relieving migraine attack symptoms and may be efficacious for treating patients suffering from migraine. Such a formulation can be prepared by adding at least one cannabinoid (i.e. THC or a derivative thereof and/or CBD or a derivative thereof) as the active pharmaceutical ingredient (API) in addition or as a replacement to cannabis oil.

Reference is now made to a formulation and a manufacturing process of a hydrophobic tablet matrix, as a further example of the composition and process of the present invention.

For the production of the hydrophobic tablet matrix a wet granulation process is applied, during which, ethanolic solution of cannabis oil and/or at least one cannabinoid is absorbed by a mix of Aerosil 972 and carnauba wax. After the steps of drying and milling, a green granulate is obtained. At the step of direct compression, mannitol, hypromellose and silica are added to improve the blend flowability. Addition of hydrophobic components is optional.

Table 4 exemplifies ingredients and production process of a hydrophobic tablet matrix containing cannabis oil.

TABLE 4
A hydrophobic tablet matrix containing cannabis oil

| Core | | | | |
|---|---|---|---|---|
| %/ | mg/ | Liquids | | |
| tablet | tablet | mg/tablet | Raw Materials | Function |
| | | | PART I Wet Granulation | |
| 20.00% | 50.00 | | Cannabis Oil | API |
| | | | (20% THC/5% CBD) | |
| | | 10.0 | Ethanol | Solvent |
| 16.00% | 40.00 | | Hydrophobic fumed silica | Absorbent |
| | | | (Aerosil 972) | |
| 8.00% | 20.00 | | Carnauba Wax | Lubricant |
| | | | | and |
| | | | | binder |
| | | | PART II Direct Compression | |
| 12.00% | 30.00 | | HPMC (Benecel E5) | Glidant |
| 25.20% | 63.00 | | Mannitol | Binder |
| 0.80% | 2.00 | | Acesulfame Potassium | Sweetener |
| 2.00% | 5.00 | | Sodium Stearyl Fumarate | Lubricant |
| | | | (Alubra) | |
| 8.00% | 20.00 | | Aerosil 200 | Glidant |
| 8.00% | 20.00 | | Sodium Crosscarmellose | Disintegrant |
| 100% | 250.00 | 10.0 | | |

The solid formulations as exemplified in Tables 3 and 4 can be formulated as sublingual tablets containing THC and/or CBD combination and administered in therapeutically amounts to patients for relieving migraine attack.

### REFERENCES

Steiner T S, Stovner L J, Birbeck G L. Migraine: the seventh disabler. Journal of Headache and Pain 2013;14:1.

Hazard E, Munakata J, Bigal M E, Rupnow M F, Lipton R B. The burden of migraine in the United States: current and emerging perspectives on disease management and economic analysis. Value in Health 2009;12(1):55-64.

Vos T, Flaxman A D, Naghavi M, Lozano R, Michaud C, Ezzati M, et al. Years lived with disability (YLDs) for 1160 sequelae of 289 diseases and injuries 1990-2010: a systematic analysis for the Global Burden of Disease Study 2010. Lancet 2012;380(9859):2163-96.

Stovner L J, Andree C. Prevalence of headache in Europe: a review for the Eurolight project. Journal of Headache and Pain 2010; 11(4): 289-99.

Victor T W, Hu X, Campbell J C, Buse D C, Lipton R B. Migraine prevalence by age and sex in the United States: a life-span study. Cephalalgia 2010;30(9):1065-72.

Ayzenberg I, Katsarava Z, Sborowski A, Chernysh M, Osipova V, Tabeeva G, et al. The prevalence of primary headache disorders in Russia: a countrywide survey. Cephalalgia 2012;32(5):373-81.

Yu S, Liu R, Zhao G, Yang X, Qiao X, Feng J, et al. The prevalence and burden of primary headaches in China: a population-based door-to-door survey. Headache 2012;52(4):582-91.

Silberstein S D. Practice parameter: Evidence-based guidelines for migraine headache (an evidence-based review): Report of the Quality Standards Subcommittee of the American Academy of Neurology. Neurology. 2000;55:754-762.

Bigal M E, Serrano D, Reed M, Lipton R B. Chronic migraine in the population: burden, diagnosis, and satisfaction with treatment. Neurology 2008;71(8):559-66.

Diamond S, Bigal M E, Silberstein S, Loder E, Reed M, Lipton R B. Patterns of diagnosis and acute and preventive treatment for migraine in the United States: results from the American Migraine Prevalence and Prevention study. Headache 2007;47(3):355-63.

Lipton R B, Bigal M E, Diamond M, Freitag F, Reed M L. Migraine prevalence, disease burden, and the need for preventive therapy. Neurology 2007;68(5):342-9.

Lucas C, Géraud G, Valade D, Chautard M H, Lantéri-Minet M. Recognition and therapeutic management of migraine in 2004, in France: results of FRAMIG 3, a French nationwide population-based survey. Headache 2006;46(5):715-25.

Radtke A, Neuhauser H. Prevalence and burden of headache and migraine in Germany. Headache 2009;49(1):79-89.

Ferrari M D, Roon K I, Lipton R B, Goadsby P J. Oral triptans (serotonin 5-HT (1B/1D) agonists) in acute migraine treatment: a meta-analysis of 53 trials. Lancet. 2001;358(9294):16.

Schnelle M, Grotenhermen F, Reif M, Gorter R W. Ergebnisse einer standardisierten Umfrage zur medizinischen Verwendung von Cannabisprodukten im deutschen Sprachraum, [Results of a standardized survey on the medical use of cannabis products in the German-speaking area] [Research in Complementary Medicine]. Forsch Komplementarmed. 1999;6(Suppl. 3):28-36.

El-Mallakh R S. Marijuana and migraine. Headache. 1987;27:442-443

Gieringer D. Medical use of cannabis: Experience in California. In: Grotenhermen F , Russo E B , eds. Cannabis and Cannabinoids: Pharmacology, Toxicology and Therapeutic Potential. Binghamton, N.Y.: The Hawthorn Press, Inc; 2002:143-152.

M Fishbein. Migraine associated with menstruation. J. Am. Med. Assoc., 237 (1942), p. 326.

Sarchielli P, Pini L A, Coppola F, et al. Endocannabinoids in chronic migraine: CSF findings suggest a system failure. Neuropsychopharmacology. 2007;32:1384-1390.

Russo E B. Clinical endocannabinoid deficiency (CECD): Can this concept explain therapeutic benefits of cannabis in migraine, fibromyalgia, irritable bowel syndrome and other treatment-resistant conditions? Neuro Endocrinol Lett. 2004;25:31-39.

Cupini L M, Bari M, Battista N, et al. Biochemical changes in endocannabinoid system are expressed in platelets of female but not male migraineurs. Cephalalgia. 2006;26:277-281.

Akerman S, Kaube H, Goadsby PJ Anandamide is able to inhibit trigeminal neurons using an in vivo model of trigeminovascular-mediated nociception. J Pharmacol Exp Ther. 2004;309:56-63

Al-Hashel J Y, Ahmed S F, Alroughani R, Goadsby P J. Migraine among medical students in Kuwait University. The Journal of Headache and Pain 2014, 15:26.

Loder E and Burch R. Measuring pain intensity in headache trials: which scale to use? Cephalalgia 2012, 32(3) 179-182.

Ferrari MD1, Goadsby P J, Roon K I, Lipton R B.Triptans (serotonin, 5-HT1B/1D agonists) in migraine: detailed results and methods of a meta-analysis of 53 trials. Cephalalgia. 2002 Oct;22(8):633-58.

1.-107. (canceled)

108. A composition comprising a therapeutically effective amount of Tetrahydrocannabinol (THC) or a derivative thereof, or Cannabidiol (CBD) or a derivative thereof, or a combination thereof in a predefined ratio conferring relief of migraine attack symptoms of a patient, wherein said migraine attack is selected from the group consisting of acute migraine attack and chronic migraine attack.

109. The composition of claim 108, wherein said CBD and said THC are in a predefined ratio conferring an additive effect with respect to relieving of migraine attack symptoms relative to the effect conferred by said CBD and said THC administered separately in a similar concentration.

110. The composition of claim 108, wherein said CBD and said THC are in a predefined ratio conferring a synergistic effect with respect to relieving of migraine attack symptoms relative to the effect conferred by said CBD and said THC administered separately in a similar concentration.

111. The composition of claim 108, wherein said predefined ratio of said CBD and said THC is selected from the group consisting of about 1:1, 1:5, 5:1, 1:4 and 4:1, respectively.

112. The composition of claim 108, wherein said symptoms are selected from the group consisting of nausea, vomiting, sensitivity to light, sensitivity to sound, sensitivity to smell and any combination thereof.

113. The composition of claim 108, wherein at least one of the following holds true:
a. said THC or a derivative thereof is selected from the group consisting of natural THC or a derivative thereof produced in the body of humans and animals, THC or a derivative thereof extracted from plants, synthetic THC or a derivative thereof, THC, THCV, THCA, THCVA and any combination thereof;
b. said THC or a derivative thereof is extracted from cannabis plant; said cannabis plant is selected from a group consisting of: Cannabis sativa, Cannabis indica, Cannabis ruderalis, and any combination thereof;
c. the concentration of said THC or a derivative thereof in said composition is in the range of about 0.2% to about 10%;
d. the concentration of said THC or said derivative thereof is in the range of about 2% (wt.) to about 20% (wt.);
e. said composition is configured for administration in a dosage unit of between about 5 mg to about 50 mg of said THC or a derivative thereof.
f. said composition comprises between about 1 wt. % to about 10 wt. % of said THC or a derivative thereof.

114. The composition of claim 108, wherein at least one of the following holds true:
a. said Cannabidiol (CBD) or a derivative thereof is selected from the group consisting of natural CBD or a derivative thereof produced in the body of humans and animals, CBD or a derivative thereof extracted from plants, synthetic CBD or a derivative thereof, CBD, CBDV, CBDA and any combination thereof;
b. said CBD or a derivative thereof is extracted from cannabis plant; said cannabis plant is selected from a group consisting of: Cannabis sativa, Cannabis indica, Cannabis ruderalis, and any combination thereof;
c. the concentration of said CBD or a derivative thereof in said composition is in the range of about 1% to about 50%;
d. said composition is configured for administration in a dosage unit of between about 1 mg to about 300 mg of said CBD or a derivative thereof;
e. said composition comprises between about 1 wt. % to about 30 wt. % of said CBD or a derivative thereof;
f. the concentration of said CBD or said derivative thereof is in the range of about 2% (wt.) to about 20%. (wt.).

115. The composition of claim 108, wherein said composition comprises cannabis oil in a concentration of about 2% (wt.) to about 25% (wt.).

116. The composition of claim 108, wherein said composition provides an improvement in migraine attack symptoms of said patient measured by at least one pain severity scale, compared to an established baseline or placebo.

117. The composition of claim 116, wherein at least one of the following holds true:
a. said improvement in migraine attack symptoms of said patient is measured by a decrease in the patient's score of at least one point or level on said at least one pain severity scale, as compared to an established baseline or to a placebo;
b. said improvement in migraine attack symptoms of said patient is measured by a decrease in the patient's score of at least two points at the first two hours from the administration of said composition to said patient, on said at least one pain severity scale, as compared to an established baseline or to a placebo;
c. said pain severity scale is selected from a group consisting of four point pain severity scale, Chronic Migraine Severity Scale, Comparative Pain Scale, Wong baker scale, The Chronic Pain Index, the Migraine Disability Assessment (MIDAS), the Headache Impact Test (HIT), the Visual Analogue Scale (VAS), the 4-point verbal rating scale (VRS), the 6-point VRS, the Numerical rating scales (NRS), the Faces Pain Rating Scale and any combination thereof;
d. said symptoms are selected from a group consisting of nausea, vomiting, sensitivity to light, sensitivity to sound, sensitivity to smell and any combination thereof.

118. The composition of claim 108, wherein at least one of the following holds true:
a. said composition is formulated for an administration route selected from the group consisting of: intranasal, transdermal, intravenous, vaginal, sublingual, buccal, oral, and any combination thereof;
b. said composition is formulated in a dosage form selected from the group consisting of liquid, solid, gas, oral, sublingual, pill, tablet, capsule, buccal, tincture, strip, film, spray, lozenge, effervescent form, sub-lingual, granules, orally-disintegrating, thin film, liquid, solution, suspension, emulsion, powder or liquid or solid crystals, powder, pastes, inhalational, aerosol, inhaler, nebulizer, smoking, vaporizer, parenteral, intradermal, intramuscular, intraos seous, intraperitoneal, intravenous, subcutaneous, topical, cream, gel, liniment or balm, lotion, ointment, drops, syrup, skin patch, vaginal, suppository, pessary, rectal, sustained release dosage form, immediate release dosage form and any combination thereof.

119. The composition of claim 108, wherein said composition is administered in combination with at least one migraine therapeutic agent selected from a group consisting of pain-relieving medications, aspirin, acetaminophen, indomethacin, Ergots, anti-nausea medications, NSAID, triptan, Opioid medications, Glucocorticoids such as prednisone and dexamethasone, Cardiovascular drugs, Antidepressants, Anti-seizure drugs, OnabotulinumtoxinA (Botox), Pain relievers and any combination thereof.

120. The composition of claim 119, wherein said composition combined with at least one migraine therapeutic agent provides a synergistic effect with respect to relieving migraine attack symptoms relative to the effect provided by said migraine therapeutic agent administered separately.

121. The composition of claim 108, wherein at least one of the following holds true:
a. said composition additionally comprises at least one carrier or excipient selected from the group consisting of diluent, solvent, absorbent, anti-adherent, binder, coatings, disintegrant, surfactant, dissolving agent, solubilizing agent, bioadhesive agent, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents, glidant, flavours, colours, sweetener, thickener, lubricant, sorbents, preservatives, and any combination thereof;
b. said composition is administered once, twice, three or four times through the day;
c. said composition is dissolved in a lipophilic solvent or suspension carrier selected from a group consisting of ethanol, medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

122. The composition of claim 108, wherein at least one of the following holds true:
a. said composition is substantially free of a pharmaceutically acceptable emulsifying agent or surfactant;
b. said THC and said CBD are formulated for penetrating the mucosal barrier;
c. said composition is formulated for rapid disintegration upon administration;
d. said composition is not significantly psychoactive.

123. The composition of claim 118, wherein said sustained release dosage form is selected from the group consisting of liposomes, drug polymer conjugates, microencapsulation, controlled-release tablet coating, and any combination thereof.

124. The composition according to claim 108, wherein administration of said composition to a patient suffering from migraine attack:
a. increases cerebral blood flow (CBF) as measure by functional magnetic resonance imaging (fMRI), in comparison with the cerebral blood flow (CBF) of said patient prior to the administration of said composition to said patient or as compared to a healthy control, and/or
b. reduces paroxysmal cortical neuron depolarization as measure by electroencephalogram (EEG) or functional magnetic resonance imaging (fMRI), in comparison with the paroxysmal cortical neuron depolarization of said patient prior to the administration of said composition to said patient or as compared to a healthy control.

125. A method for relieving migraine attack of a patient comprising steps of
a. providing a composition comprising Tetrahydrocannabinol (THC) or a derivative thereof, or Cannabidiol (CBD) or a derivative thereof, or a combination thereof; and
b. administering said composition to said patient at a therapeutically effective dosage for relieving migraine attack of said patient.

126. A method for increasing cerebral blood flow (CBF) in a patient suffering from migraine attack, said method comprises steps of:
a. administering the composition according to claim 108 to said patient at a therapeutically effective dosage; and
b. detecting increase in cerebral blood flow (CBF) of said patient by functional magnetic resonance imaging (fMRI) analysis.

127. A method for reducing paroxysmal cortical neuron depolarization in a patient suffering from migraine attack comprising steps of
a. administering to said patient the composition according to claim 108 at a therapeutically effective dosage; and
b. detecting decrease in paroxysmal cortical neuron depolarization of said patient by electroencephalogram (EEG) or functional magnetic resonance imaging (fMRI) analysis.

128. A composition comprising a therapeutically effective amount of Cannabidiol (CBD) or a derivative thereof or a Tetrahydrocannabinol (THC) or a derivative thereof for use in relieving migraine attack of a patient, wherein said composition is prepared by steps of:
a. preparing a mixture comprising an effective amount of cannabis oil, by a wet granulation process; and,
b. formulating said mixture in a solid dosage form by direct compression.
